(19) United States
(12) Patent Application Publication
(10) Pub. No.: US 2014/0302027 A1
(43) Pub. Date: Oct. 9, 2014

(54) METHODS OF TREATING PERIODONTAL INFLAMMATION AND PERIODONTAL BONE LOSS

(71) Applicants: United States of America as Represented by the Secretary, Dept. of Health and Human Services, Bethesda, MD (US); University of Louisville Research Foundation, Inc., Louisv ille, KY (US)

(72) Inventors: George Hajishengallis, Philadelphia, PA (US); Triantafyllos Chavakis, Dresden (DE)

(73) Assignee: UNIVERSITY OF LOUISVILLE RESEARCH FOUNDATION, INC., Louisville, KY (US)

(21) Appl. No.: 14/347,434

(22) PCT Filed: Sep. 26, 2012
(86) PCT No.: PCT/US2012/057329

§ 371 Date: Mar. 26, 2014

Related U.S. Application Data
(60) Provisional application No. 61/539,315, filed on Sep. 26, 2011.
(60) Provisional application No. 61/602,413, filed on Feb. 23, 2012.

(51) Int. Cl. C07K 14/47; G01N 33/53; A61K 35/12

(52) U.S. Cl. CPC C07K 14/47; A61K 35/12; G01N 33/53; G01N 2800/10
USPC 424/134.1; 514/21.2; 424/93.7; 514/16.7; 56/9

(57) ABSTRACT

Methods and compositions for use in treating periodontitis include a Del-1 polypeptide.

### RELATED APPLICATIONS

This application is related to U.S. Provisional Application Ser. Nos. 61/539,315 filed Sep. 26, 2011 and 61/602,413 filed Feb. 23, 2012, the entire disclosures of which are incorporated herein by this reference.

### GOVERNMENT INTEREST

This invention was made with government support under RO1 DE018292, DE021580, DE015254, DE017138, and DE021685 awarded by the National Institutes of Health. The United States government has certain rights in the invention.

### TECHNICAL FIELD

The presently-disclosed subject matter relates to treatment of periodontal disorders. In particular, the presently-disclosed subject matter relates to treatment of periodontal inflammation and/or bone loss by administering a Del-1 polypeptide.

### INTRODUCTION

Advanced age is associated with a number of chronic inflammatory diseases, including an increased prevalence and severity of periodontitis. This oral disease affects the majority of the adult population, and an estimated 10-15% develops severe periodontitis, which not only leads to tooth loss but is also a risk factor for systemic conditions, such as atherosclerosis, rheumatoid arthritis, adverse pregnancy outcomes, diabetes, and aspiration pneumonia. In addition to the aging adult population, individuals with conditions or syndromes affecting neutrophil recruitment to peripheral tissues rapidly develop severe periodontitis early in life. Neutrophil dysfunction-associated periodontitis in children leads to loss of primary and permanent teeth, causing serious adverse psychological and functional consequences (esthetics, mastication, and speech).

Conventional periodontal treatment is often insufficient to reverse destructive inflammation, and many patients develop recurrent disease for reasons that are not clear. This necessitates better understanding of the underlying immunopathology and improved therapeutic approaches, especially in the elderly who are more susceptible to periodontitis. Indeed, despite intensive research, effective topical formulations for the control of periodontal inflammation are currently lacking. The annual cost of periodontal therapy in the United States exceeds $14 billion and the link between periodontal disease and certain systemic diseases underlines the gravity of this chronic inflammatory disease of the oral cavity. Accordingly, there remains a need in the art for methods and compositions useful for the treatment of periodontal inflammation, bone loss, and/or periodontitis.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes methods, compositions, and kits useful for treating periodontal inflammation, bone loss, and/or periodontitis. Methods of the presently-disclosed subject matter involve administering to a subject a Del-1 polypeptide. Compositions and kits of the presently-disclosed subject matter include a Del-1 polypeptide.

In some embodiments, a method of the presently-disclosed subject matter includes administering an effective amount of a Del-1 polypeptide, as disclosed herein. In some embodiments, the method can also involve monitoring the subject for periodontal inflammation and/or bone loss. In some embodiments, one or more markers associated with periodontal inflammation and/or bone loss can be monitored. Examples of such markers include, but are not limited to, IL-1β and MMP-8. Embodiments of the method can also include identifying a subject having periodontitis or a risk thereof. In some embodiments, the subject is identified as having periodontal inflammation and/or bone loss.

In some embodiments of the presently-disclosed subject matter, the method includes administering the Del-1 polypeptide to a subject having a condition associated with neutrophil dysfunction. In some embodiments, the subject has a condition selected from: a leukocyte adhesion deficiency (LAD), Wiskott-Aldrich syndrome, Chediak-Higashi syndrome, lazy leukocyte syndrome, Papillon-LeFevre syndrome, Down's syndrome, congenital agranulocytosis, cyclic neutropenia (idiopathic or drug-induced), autoimmune neutropenia, chronic idiopathic neutropenia, HIV-associated neutropenia, and neutropenia in cancer patients under chemotherapy and/or radiation therapy.

Embodiments of the method can include administering the Del-1 polypeptide to a subject of a certain age. In some embodiments, the subject is at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years old.

The Del-1 polypeptide can be administered to the mouth of the subject. In some embodiments, for example, the Del-1 polypeptide can be administered to the gingiva and/or periodontal pocket of the subject. The Del-1 polypeptide can be administered periodically. For example, in some embodiments, the Del-1 polypeptide is administered at least 4 times daily, 3 times daily, 2 times daily, daily, every other day, every third day, every fourth day, every fifth day, every sixth day, or weekly. In some embodiments, the Del-1 polypeptide is administered at least daily. The periodic administration can occur during a treatment period. In some embodiments, the treatment period can last at least 1, 2, 3, 4, 5, 6, 7, 8, 9, 10, 11, or 12 months. In some embodiments, the treatment period is indefinite, e.g., administration in connection with regular oral hygiene during the lifetime of the subject.

The presently-disclosed subject matter includes a composition that comprises a Del-1 polypeptide formulated for delivery to an oral cavity/mouth of a subject. The composition can include a vehicle in which the Del-1 polypeptide is provided. The vehicle is appropriate for delivery to the oral cavity of the subject.

In some embodiments of the presently-disclosed subject matter, the Del-1 polypeptide is provided as a fusion protein. The Del-1 polypeptide can be provided as a fusion protein that includes an IgG Fc fragment.

In some embodiments of the presently-disclosed subject matter, the Del-1 polypeptide is provided in a toothpaste, a mouthwash, a chewing gum, a dental floss, a beverage, a food product, a gel, a slow-release gel, a tablet, a granule, a film, or a thin film of biodegradable matrix.

In some embodiments of the presently-disclosed subject matter, the Del-1 polypeptide is expressed by a cell containing a nucleic acid encoding the Del-1 polypeptide, which cell can be administered to a subject.

In some embodiments of the presently-disclosed subject matter, the Del-1 polypeptide is used in the treatment of a chronic inflammatory conditions, adverse pregnancy outcomes, aspiration pneumonia, atherosclerosis, chronic obstructive pulmonary disease, diabetes, inflammatory bowel diseases, and/or rheumatoid arthritis.

The presently-disclosed subject matter further includes a kit. In some embodiments, the kit includes a Del-1 polypeptide and a device for administering the Del-1 polypeptide or composition. Appropriate devices will be recognized by one or ordinary skill in the art, but can include, for example, a syringe or a toothbrush. In some embodiments, a kit can include multiple doses of the Del-1 polypeptide. In some embodiments, the multiple doses are included in a packet of chewing gum.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Mouse model for induction of periodontal bone loss. Antibiotics are used to transiently suppress the normal oral flora to facilitate oral infection with P. gingivalis (10⁹ CFU in 2% carboxymethylcellulose vehicle). In the standard protocol, using young mice, the animals are sacrificed six weeks after the last infection (day 64) for determination of bone loss in defleshed maxillae (Hajishengallis (2007), Armitage (2002)).

FIG. 2. Major inflammatory parameters in the mouse model of periodontitis and therapeutic approaches. Induction of inflammatory mediators in periodontal tissue of mice infected with P. gingivalis determined by quantitative real-time PCR (A) and images of periodontal bone loss, which is determined morphometrically (B). In panel C, local gingival injections of a CR3 antagonist (XVA143) inhibited P. gingivalis (Pg)-induced bone loss. (D) Oral infection of mice with P. gingivalis causes a major increase in the numbers of their indigenous oral anaerobic flora.

FIG. 3. Old mice develop periodontal bone loss and oral infection with P. gingivalis accelerates the process. (A) Increased periodontal bone loss with increasing mouse age, measured as the distance between the cementoenamel junction (CEJ) and the alveolar bone crest (ABC) (Niederman (2001)). (B) 15-month-old mice were orally infected or not with P. gingivalis and CEJ-ABC distance measurements were performed 6 weeks after infection, following mouse euthanasia. The baseline values (2.3±0.26) were determined using an independent group of 15-month-old mice. The data are means±SD (n=5 mice/group). *, p<0.01.

FIG. 4. Del-1 is expressed in mouse gingiva and expression is reduced in old age. (A) Quantitative real-time PCR was used to determine Del-1 mRNA expression levels (normalized against GAPDH mRNA levels) in the indicated tissues (brain and liver served as positive and negative controls, respectively) obtained from 8-10 week-old C57BL/6 mice. (B) Expression of Del-1 protein in the same tissues, determined by immunohistochemisty, using rabbit IgG antibody to mouse/human Del-1, and visualized by confocal microscopy. Shown are representative single optical sections (left), differential interference contrast (DIC) images (middle), and overlay (right). (C) Relative expression of Del-1 mRNA in the gingiva of young (8-10 weeks old) and old (≧18 months) mice, determined as in A, after normalization to indicate fold difference of young compared to old. (D) Visualization of Del-1 protein in inderdental gingiva of young and old mice using confocal microscopy as in B. Numerical data are means±SD (n=5).

FIG. 5. Decreased periodontal expression of Del-1 in old mice correlates with increased neutrophil influx. C57BL/6 mice (8-10 weeks of age [young; Y] or ≧18 months of age [old; O]) were orally infected with P. gingivalis. After 24 h, the mice were euthanized and the upper jaws were processed for immunohistochemistry. Mesio-distal sections parallel to the long axis of the teeth were stained with specific antibodies to Del-1 and LyG6 (neutrophil marker) followed by incubation with fluorescently-labeled secondary antibodies. (A) Images were captured using a confocal microscope (Olympus V500. Shown are representative fluorescence/DIC overlay images. In addition to the gingiva, the pulp is also positive for Del-1. (B) Florescence intensity for Texas red (Del-1) or FITC (LyG6) in the demarcated gingival areas was calculated using the ImageJ software. The images and relative fluorescent intensity values are typical of five mice used per treatment group.

FIG. 6. Del-1 deficiency leads to inflammatory periodontal bone loss. (A) C57BL/6 wild-type or Del-1^{-/-} mice were orally infected or not with P. gingivalis and evaluated for induction of periodontal bone loss. CEJ-ABC distance values were transformed to directly indicate bone loss, as previously described (Armitage (2002)). (B) Gingiva were excised from the same groups and processed for quantitative real-time PCR to determine mRNA expression levels for the indicated molecules (normalized against GAPDH mRNA levels and expressed as fold induction relative to wild-type, sham-infected group). Data are means±SD (n=5 mice). *, p<0.01 vs. wild-type sham-infected. •, p<0.01 vs. all other groups.

FIG. 7. Del-1 expressed as an Fc fusion protein, which may increase its bioavailability in the tissue, was microinjected in the palatal gingiva of the ligated second molar, one day before placement of the ligature and every day thereafter until the day before sacrifice (day 5), leading to significant inhibition of bone loss (p<0.01). In a parallel experiment utilizing the same protocol, treatment with a neutralizing monoclonal antibody (mAb) to IL-17 also resulted in significant inhibition of bone loss. In contrast, treatment with IgG2a (Fc control and isotype control for the anti-IL-17 mAb) was without any effect.

FIG. 8. Del-1 is expressed in the mouse gingiva by endothelial cells. (a) Brains, livers, and gingiva were harvested from 8-week-old C57BL/6 mice and processed for quantitative real-time PCR (qPCR) to determine Del-1 mRNA expression (normalized against GAPDH mRNA; data are means±SD [n=3 mice per group] from one of two independent experiments that yielded similar results. Brain and liver served as positive and negative control, respectively, for gingiva. (b) Sagittal sections of interdental gingiva were stained for Del-1 and CD31 (endothelial cell marker), as indicated, with colocalization (arrows) shown in the merged image (scale bar, 50 μm). The lower row contains the overlays of the same fluorescent confocal images with corresponding DIC images. (c) X-gal staining of gingiva from Edil3^{-/-} mice (left panel); these mice are Del-1 knock-out/LacZ knock-in transgenics where the LacZ gene is controlled by the native Del-1 promoter and is thus a reporter for Del-1 expression. The relatively restricted pattern of X-gal-staining in the connective tissue is contrasted with Del-1 staining in the confocal image (middle panel) involving both the connective tissue and the epithelium (unlike Del-1, LacZ-encoded β-galactosidase is not secreted). As expected, no positive X-gal staining was detected in wild-type control mice (right panel). (d) Gingiva were stained for β-galactosidase and CD31, as indicated, with colocalization shown in the merged image; shown are overlays of DIC and fluorescent confocal images (scale bar, 50 μm). The similar expression pattern of β-galactosidase (reporter for local Del-1 expression) and CD31 (endothelial cell marker) indicates that Del-1 is expressed by endothelial cells in the mouse gingiva. The immunohistochemical analysis is representative of 5 (b) or 6 (c-d) mice.

FIG. 9. Reduced expression of Del-1 in old mice correlates with periodontal bone loss. (a) Increased bone loss in old C57BL/6 mice (18 months) compared to young controls (8-10 weeks) (inset), calculated based on measured CEJ-ABC distances. Pooled data from two independent experiments (n=10 mice per group). (b) Gingiva were dissected from the same mice and qPCR was used to determine Del-1 mRNA expression (normalized against GAPDH mRNA and expressed as fold change of old relative to young mice, the average value of which was taken as 1). (c) Sagittal sections of interdental gingiva were stained for Del-1 protein or the neutrophil marker Ly6G; shown are representative overlays of differential interference contrast (DIC) and fluorescent confocal images (bar, 50 μm; T, tooth; G, gingiva; S, sulcus). (d) The fluorescence intensities of these and additional representative images from independent mice were quantified using ImageJ analysis (data are means±SD; n=5 mice per group, 3 mice each from the first experiment and 2 mice each from the second experiment which were combined in a). (e-f) Linear-regression analysis of the CEJ-ABC distance values versus Del-1 expression in old (e) and young (f) mice using the data from a,b. *P<0.01.

FIG. 10. Del-1 deficiency is associated with inflammatory periodontal bone loss and neutrophil infiltration. (a) Sixteen-week-old C57BL/6 wild-type (WT) or Edil3^{-/-} mice, of either gender, were assessed for periodontal bone heights (upper panel); data were transformed to indicate bone loss in Edil3^{-/-} mice relative to WT bone heights (lower panel). (b) Gingiva were dissected from 16-week-old WT or Edil3^{-/-} mice and mRNA expression of the indicated molecules was determined by qPCR (normalized against GAPDH mRNA and expressed as fold change in Edil3^{-/-} transcript abundance relative to WT). (c,d,e) Sagittal sections of maxillary teeth from 16-week-old WT or Edil3^{-/-} mice were stained for IL-17A (c) or the neutrophil marker Ly6G (e). Shown are representative fluorescent confocal images [left] and corresponding DIC images [right] (T, tooth; G, gingiva; S, sulcus). The fluorescence intensities of these and additional representative images from independent mice (5 per group) were quantified using ImageJ analysis (d). (f) Time course of bone loss in Edil3^{-/-} mice and WT littermate controls; negative values indicate bone loss relative to 5-week-old WT. Data are means±SD (n=4-6 mice per group) from one of two independent sets of experiments yielding similar results. *P<0.05; **P<0.01 compared to corresponding control.

FIG. 11. Del-1 deficiency is associated with increased osteoclastic activity in the periodontium. (a) Sagittal sections of maxillary teeth from 20-week-old wild-type (WT) or Edil3^{-/-} mice were stained for RANKL (scale bar, 50 μm; T, tooth; G, gingiva; B, bone). Shown are representative overlays of DIC and fluorescent confocal images (upper panels). There is increased RANKL staining in regions adjacent to the periodontal bone in Edil3^{-/-} mice relative to wild-type controls. In the lower panels, the images were also stained with DAPI to visualize cell nuclei. Images are representative of 5 mice (b) Representative histological sections (n=5 mice) showing increased numbers of TRAP-positive cells (arrows) in the periodontal bone of 20-week-old Edil3^{-/-} mice as compared to wild-type controls (original magnifications ×200 & ×400 in the upper and lower panels, respectively). (c) The RANKL fluorescence intensities of images shown in A, and of additional representative images from independent mice (5 per group) were quantified using ImageJ analysis. (d) Mean number (±SD) of TRAP-positive cells determined in 10 random microscopic fields, including those shown in B (original magnification ×200). (e) Total and trabecular bone mineral density (BMD) was measured at the distal femur or the fourth lumbar vertebrae of WT or Edil3^{-/-} mice by peripheral quantitative computer tomography. Numerical data represent means (±SD; n=5-6 mice per group). *P<0.05; **P<0.01 compared to corresponding control.

FIG. 12. Increased mRNA expression of inflammatory mediators in the periodontium of Edil3^{-/-} mice. Gingiva were dissected from wild-type (WT) or Edil3^{-/-} mice at the age of 8 weeks (a) or 9 months (b) and gingival mRNA expression of the indicated molecules was determined by qPCR (normalized against GAPDH mRNA and expressed as fold change in Edil3^{-/-} transcript abundance relative to that of WT, the average value of which was taken as 1). Data are means±SD (n=4 mice per group). *p<0.05; **p<0.01 compared to corresponding WT control.

FIG. 13. Edil3^{-/-} mice harbor higher numbers of oral bacteria which are required for periodontal bone loss. (a) Wild-type (WT) and Edil3^{-/-} mice at the indicated ages were assessed for oral anaerobic bacteria by culture; CFU are shown for each mouse with horizontal lines denoting mean values. (b) Total bacteria were enumerated in the periodontal tissue of WT and Edil3^{-/-} mice at the indicated ages by quantitative real-time PCR of the 16S rRNA gene. Data are means±SD (n=5 mice per group). (c) Edil3^{-/-} mice were given antibiotics (sulfamethoxazole and trimethoprim) in the drinking water since weaning (plain water served as control; ‘untreated’) and after 14 or 20 weeks were assessed for bone loss (relative to a baseline determined by 5-week-old Edil3^{-/-} mice) Data are means±SD (n=5 mice per group). *p<0.05; **p<0.01 compared to corresponding WT control. ^{†}At 5 weeks, Edil3^{-/-} mice have not yet started experiencing bone loss and their bone heights are similar to those of WT littermate controls (see FIG. 10f).

FIG. 14. LFA-1 dependence of Del-1 deficiency-associated inflammation and bone loss. (a) Twenty-week-old C57BL/6 wild-type (WT) mice or mice deficient in the indicated molecules were assessed for bone loss relative to 5-week-old WT mice (zero baseline); the dotted line indicates the bone heights of 20-week-old WT mice for ease of comparison. (b) Sagittal sections of interdental gingiva from the same mice were stained for Ly6G; shown are representative overlays of DIC and fluorescent confocal images. (c) The fluorescence intensities of these and additional representative images from independent mice (5 per group) were quantified using ImageJ analysis. (d) Dissected gingiva from the indicated mice were processed for ELISA determination of MPO protein (left) or qPCR determination of PGRP1 transcript abundance (right). (e) Transmigration of human neutrophils through an endothelial cell monolayer (HUVEC) towards IL-8 (20 ng/ml) in the bottom well was determined with or without soluble human Del-1 (5 μg/ml), BSA (5 μg/ml), anti-LFA-1 mAb (10 μg/ml) or IgG1 control (10 μg/ml). Transmigration without inhibitors (or controls) was set to 100% (dashed line). (f) Sections of interdental gingiva from the indicated mice were stained for IL-17 (left) and Ly6G (middle) with colocalization shown in merged images (right); the bottom row includes magnified views of the demarcated areas. Data are means±SD (a,c,d, n=5; e, n=4) from one of two to four independent experiments yielding similar results. *P<0.01. Scale bars, 50 μm.

FIG. 15. Mouse neutrophils express IL-17A and their recruitment in the gingiva increases with advancing age. (a-b) Mouse neutrophils isolated from the bone marrow were treated with medium only (unstimulated) or stimulated with PMA (20 ng/ml) for 4 h (a) or with PMA (20 ng/ml) plus ionomycin (1 μg/ml) for 4 h (b) and assessed for IL-17A mRNA expression or IL-17A protein release, respectively (TNF determinations served as positive controls). The mRNA expression data are from one representative experiment (out of four independent experiments yielding similar results). The protein data are means±SD (n=3 sets of neutrophils per group) from one of three independent experiments that yielded similar results. (c) Dissected gingiva from Edil3^{-/-} mice, of the indicated ages, were processed for ELISA determination of MPO protein amounts. MPO served as a marker of neutrophil infiltration. Data are means±SD (n=6 mice per group). *P<0.01 compared to 8-week-old group.

FIG. 16. IL-17 expression in Edil3^{-/-} gingiva by CD4⁺ or γδ-TCR⁺ cells. (a) Sections of interdental gingiva from Edi/3^{-/-} mice were stained for IL-17A and CD4, as indicated, with colocalization (arrows) shown in merged images (scale bar, 20 μm). (b) Sagittal sections of interdental gingiva from 20-week-old wild-type (WT) or Edil3^{-/-} mice were stained for CD4; shown are representative overlays of DIC and fluorescent confocal images (scale bar, 50 μm; T, tooth; G, gingiva; S, sulcus). (c) The fluorescence intensities of the images shown in b and of additional representative images from independent mice were quantified using ImageJ analysis. (d) Gingiva were dissected from WT or Edil3^{-/-} mice and gingival mRNA abundance of CD4 and PGRP-1 (neutrophil marker) was determined by qPCR (normalized against GAPDH mRNA and expressed as fold change in Edil3^{-/-} transcript abundance relative to wild-type transcript abundance, which was assigned an average value of 1). (e) Sagittal sections of interdental gingiva from 20-week-old WT or Edil3^{-/-} mice were stained for IL-17A and γδ TCR, as indicated, with colocalization (yellow) shown in merged images (scale bar, 20 μm). (f) The fluorescence intensities of the images shown in e and of additional representative images from independent mice were quantified using ImageJ analysis. Numerical data are means±SD (n=5 to 6 mice per group).*, P<0.01. The immunohistochemical analysis (a-c & e-f) is representative of 5 independent mice per group and the qPCR experiment (d) was repeated twice yielding similar results. The increased expression of the neutrophil marker PGRP-1 (d) correlates with other findings, described herein, showing increased numbers of neutrophils (confocal microscopy) and elevated MPO amounts (ELISA), whereas the comparable CD4 expression in WT and Edil3^{-/-} mice (d) is consistent with the findings from the confocal microscopy analysis (c).

FIG. 17. Del-1 deficiency-associated inflammatory bone loss is prevented in mice with dual Del-1-IL-17R deficiency. (a) Twenty-week-old C57BL/6 wild-type (WT) mice or mice with the indicated genetic deficiencies were assessed for periodontal bone heights (for details see FIG. 14a legend). (b) Five-week-old WT mice or mice with the indicated deficiencies were assessed for periodontal bone heights. (c) The mice in a were assessed for numbers of oral anaerobic bacteria. (e) Changes to the composition of the oral microbiota detected by aerobic (left) or anaerobic (right) culture in mice with single or combined Del-1 deficiencies and their wild-type littermate controls. CFU for each organism are shown as a proportion of the total cultured organisms. Data are means±SD (n>6 mice per group) from one of two independent sets experiments that yielded similar results. *P<0.01 compared to WT control.

FIG. 18. Mice with combined Del-1/IL-17R (Edil3^{-/-}Il17ra^{-/-}) or Del-1/LFA-1 (Edil3^{-/-}Itgal^{-/-}) deficiency are protected from periodontal bone loss. Representative images of maxillae (upper jaws) showing that the bone gap (CEJ-ABC distance; see cartoon) is greatest in 20-week-old mice with single Del-1 deficiency, whereas age-matched mice with combined Del-1 deficiencies have bone heights comparable to those of wild-type (WT) mice. The images are representative of the mice used in the experiment shown in FIG. 17a.

FIG. 19. Periodontal bone heights in Il17ra^{-/-} mice relative to age-matched wild-type controls. (a) C57BL/6 Il17ra^{-/-} mice and age-matched wild-type (WT) controls were assessed for periodontal bone heights. Negative values denote bone loss relative to the bone heights of 5-week-old WT mice (zero baseline); the dotted line indicates the bone heights of 20-week-old WT mice. (b) C57BL/6 WT mice or mice genetically deficient in the indicated molecules were assessed for periodontal bone heights at 30 weeks of age. Negative values denote bone loss relative to the bone heights of 5-week-old WT mice (zero baseline); the dotted line indicates the bone heights of 30-week-old WT mice for ease of comparison with age-matched gene knockout mice. Data are means±SD (a, n=7-10 mice per group; b, n=5-6 mice per group) from one of two independent sets of experiments that yielded similar results. *, P<0.05; **, P<0.01 compared to age-matched WT littermate control.

FIG. 20. Inflammatory and microbiological changes in the periodontium after anti-inflammatory treatment. To induce periodontal inflammation, silk ligatures were tied around the second molar teeth of 10-week-old wild-type mice. One such group was treated daily with meloxicam (1 mg/Kg; s.c), a nonsteroidal anti-inflammatory drug that selectively inhibits COX-2, and another group received PBS control. A third group was not ligated or received treatment and served as baseline control. After 5 days, the mice were sacrificed and assessed for MPO amounts in dissected gingiva (a) and for numbers of oral anaerobic bacteria (b). In a, data are means±SD (n=5 mice per group) In b, CFU are shown for each individual mouse with horizontal lines denoting mean values. *P<0.01 between indicated groups. The data are from one of two independent experiments that yielded consistent results. These data indicate that anti-inflammatory treatment can reduce the oral bacterial load even though it also reduced neutrophil infiltration, as indicated by diminished gingival MPO amounts.

FIG. 21. IL-17 downregulates Del-1 expression. (a) Gingival Del-1 mRNA expression in wild-type (WT) and IL17ra^{-/-} mice determined by qPCR. (b) Sagittal sections of interdental gingiva of WT and Il17ra^{-/-} mice were stained for Del-1; shown are representative fluorescent confocal images (upper row) and their overlays with corresponding DIC images (lower row). Bar, 50 μm; T, tooth; G, gingiva; S, sulcus. (c) The fluorescence intensities of these and additional representative images from independent mice (5 per group) were quantified using ImageJ analysis. (d) Anti-IL-17A mAb and IgG2a control were microinjected into the gingiva and Del-1 mRNA expression was determined by qPCR. (e) Gingival IL-17A mRNA expression was determined in young (8-10 weeks) and old (18 months) mice using qPCR. (f) Linear-regression analysis of Del-1 versus IL-17A expression in old mice. (g) Determination of Del-1 mRNA expression and MPO amounts in the gingiva of indicated BM chimeric mice (donor BM→lethally irradiated recipient). (h) HUVEC were stimulated with or without human IL-17A and Del-1 mRNA expression was quantified by qPCR (pooled data from four independent experiments). Mouse data are shown for each individual animal in scatter plots or represent means±SD (n=5 mice) in bar graphs, and are representative of two or three independent experiments. *P<0.05; **P<0.01.

FIG. 22. Expression of Del-1 and IL-17A in gingival biopsy samples from periodontitis patients. Gingival Del-1 (a) and IL-17A (b) mRNA expression in healthy and diseased sites were determined by qPCR (normalized against GAPDH mRNA and expressed as fold change relative to healthy-site transcript abundance, which was assigned an average value of 1). The pair of data for each individual is connected by a line (n=7 individuals). See Examples section describing methods for selection of patients and healthy plus diseased gingival sites. Healthy sites expressed on the average 6.5-fold higher Del-1 mRNA and 17.9-fold lower IL-17A than diseased (inflamed) sites. Both differences were statistically significant (P<0.05). These data are consistent with findings in the mouse model (described herein) that Del-1 and IL-17A expressions are inversely associated in the periodontium.

FIG. 23. Increased IL-17A expression and reduced neutrophil infiltration in the gingiva of Il17ra^{-/-} mice. Dissected gingiva from C57BL/6 wild-type (WT) or Il17ra^{-/-} mice, of the indicated ages, were processed for qPCR determination of IL-17A and PGRP1 transcript abundance (a) or ELISA determination of MPO protein amounts (b). The mRNA expression was normalized against GAPDH mRNA and expressed as fold change in Il17ra^{-/-} transcript abundance relative to wild-type transcript abundance, which was assigned an average value of 1. PGRP1 and MPO both served as markers of neutrophil infiltration. Data are means±SD (n=5-6 mice per group). *P<0.01 compared to corresponding WT control.

FIG. 24. Del-1 inhibits IL-17 and periodontal inflammation in old mice. Eighteen-month-old C57BL/6 mice were microinjected in the gingiva with BSA (control) or Del-1, as indicated. In addition, the mice were orally administered P. gingivalis in 2% carboxymethylcellulose vehicle (a, b, c; lower rows) or vehicle control (a, b, c; upper rows) and were sacrificed 12 h later. Sagittal sections of interdental gingiva were stained for the neutrophil marker Ly6G (a), IL-17A (b), or TNF (c). Shown are typical fluorescent confocal images (left) and their overlays with corresponding DIC images (right). (d) The fluorescence intensities of these and additional representative images from independent mice were quantified using ImageJ analysis; data were expressed as % intensity of the Del-1-treated groups relative to the BSA-treated controls, the value of which was set to 100% (dashed line). ‘Induced inflammation’ refers to the groups inoculated with P. gingivalis. Data are means±SD (n=5 mice per group) from one of two independent experiments yielding similar results. **P<0.01 compared to BSA-treated controls.

FIG. 25. Inflammatory host responses in ligature-induced periodontitis. Ligature-induced periodontal inflammation in C57BL/6 mice was monitored in dissected gingiva processed for determining MPO amounts by ELISA (a) or for qPCR to determine mRNA expression of the indicated molecules, normalized against GAPDH mRNA (b). The data are expressed as fold change in the transcript abundance in the ligated side relative to that of the unligated side, which was assigned an average value of 1. Data are means±SD (n=5-6 mice per group) from one of three independent experiments yielding similar results. *P<0.01 compared to corresponding control.

FIG. 26. Inhibition of bone loss by Del-1 and other treatments that block LFA-1 or IL-17. Ligature-induced periodontal bone loss was assessed in mice treated as follows: (a) 18-month-old (left) and 10-week-old mice (right) were microinjected in the gingiva with Del-1-Fc or Fc control (1 μg) or with anti-IL-17A mAb or isotype control (1 or 10 μg). (b) WT and Itgal^{-/-} mice received Del-1-Fc or Fc control as above. (c) LFA878 was locally administered at an equal molar concentration with Del-1-Fc (corresponding to 8.4 ng and 1 μg, respectively) or at the indicated higher doses. (d) Anti-LFA-1 or IgG2a control were microinjected at either 1 μg, as was Del-1-Fc, or at 10 μg. (e) Del-1-Fc, anti-LFA-1, anti-ICAM-1, ICAM-1-Fc and their controls were i.v. administered at 50 μg. Data are means±SD (n=5-6 mice per group) from one of two (a,c,e) or three (b,d) independent and representative experiments. Data represent % inhibition of bone loss, which was calculated using the formula: ([Bone loss in the absence of inhibitor−Bone loss in the presence of inhibitor]/Bone loss in the absence of inhibitor)×100. *P<0.05; **P<0.01. NS, not significant.

FIG. 27. Effects of Del-1 deficiency or Del-1 on neutrophil function. (a) Migration of neutrophils isolated from wild-type (WT) or Edil3^{-/-} mice was tested in a transwell system. Chemokinesis (in the absence of a chemokine in the bottom well) or chemotaxis to CXCL2 (10 ng/ml) in the bottom well was assessed after 60 min. The migration of WT neutrophils in the absence of chemokine was set as the 100% control (dashed line) and the data were expressed relative to this value. (b) Neutrophils isolated from WT or Edil3^{-/-} mice were stimulated or not for 4 h with LPS (100 ng/ml) plus C5a (10 nM) and release of the indicated cytokines or chemokines in the culture supernatants was determined by ELISA. (c) WT neutrophils, in the presence or absence of soluble Del-1 (10 μg/ml), were stimulated as above and assayed for release of the indicated cytokines or chemokines. Data are means±SD (n=3 sets of neutrophils), from one of two independent sets of experiments yielding similar results. (a-b) No significant differences were found in the capacities of neutrophils from WT or Edil3^{-/-} mice for migration or induction of TNF or CXCL1 release. (c) No significant differences were observed in the release of TNF or CXCL1 by neutrophils upon Del-1 treatment.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes methods, compositions, and kits useful for treating periodontal inflammation, bone loss, and/or periodontitis. Methods of the presently-disclosed subject matter involve administering to a subject an effective amount of a Del-1 polypeptide. Compositions and kits of the presently-disclosed subject matter include a Del-1 polypeptide.

Del-1 has been studied in various contexts; for example, Del-1 has been implicated in connection with effects on bone that are mediated through Del-1 action on chondrocytes (cartilage), mostly during embryogenesis and development. These prior studies might have relevance in the context of conditions involving the joints (which have chondrocytes/cartilage) such as osteoarthritis; however, there are no chondrocytes/cartilage in the periodontal bone and thus, notwithstanding such prior studies, there was nothing to suggest that Del-1 might be useful for the treatment of periodontal disorders. In this regard, Del-1 has not heretofore been suggested in connection with methods of treating periodontal inflammation and periodontal bone loss, or a risk thereof.

Developmental endothelial locus (Del)-1, which is also known as epidermal growth factor (EGF)-like repeats and discoidin-1-like domains 3 (EDIL3) will be recognized by the skilled artisan as an endothelial-cell-expressed glycoprotein, that was originally described for its role in vascularization. Del-1 can become associated with the endothelial cell surface by binding to surface proteoglycans or to integrin αvβ3 via an Arg-Gly-Asp (RGD) motif. As disclosed herein, the present inventors have found Del-1 to be useful in the treatment of periodontal inflammation, bone loss, and/or periodontitis. Periodontitis is a chronic inflammatory diseases, which leads to the destruction of the tissues that surround and support the teeth (peridodontium) and constitutes a risk factor for systemic diseases, including, for example, atherosclerosis, rheumatoid arthritis, adverse pregnancy outcomes, diabetes, and aspiration pneumonia.

In some embodiments, a Del-1 polypeptide can be a polypeptide encoded by a nucleotide sequence known to one of ordinary skill in the art, including those set forth in publicly-available databases, for example, the nucleotide sequences of accession numbers: NM_{—}005711 (2,974 bp linear mRNA) or U70312 (1719 by linear mRNA). In some embodiments, a Del-1 polypeptide can be a polypeptide having an amino acid sequence known to one of ordinary skill in the art, including those set forth in publicly-available databases, for example, the amino acid sequences of accession numbers: NP_{—}005702 (480 amino acids), O43854 (480 amino acids), or AAC02648 (480 amino acids). In some embodiments, a Del-1 polypeptide can be a polypeptide having an amino acid sequence of accession numbers: NP_{—}034233 (470 amino acids), NP_{—}001033076 (480 amino acids), O35474 (480 amino acids), or AAB86585 (480 amino acids).

The polynucleotides and polypeptides disclosed herein with reference to a GENBANK®/GENPEPT® accession number, or other accession number associated with a publicly-available database, are cross-referenced to the sequence of the publicly-available database, and such sequences in the publicly-available database are expressly incorporated herein by reference. Also expressly incorporated herein by reference are all annotations present in the publicly-available database associated with the accession numbers and sequences disclosed herein. Unless otherwise indicated or apparent, the references to the publicly-available databases are references to the most recent version of the databases as of the filing date of this Application.

In some embodiments, the Del-1 polypeptide can be a polypeptide comprising the sequence of SEQ ID NO: 1. In some embodiments, the Del-1 polypeptide can be a polypeptide encoded by the nucleic acid of SEQ ID NO: 2.

The terms “nucleotide,” “polynucleotide,” “nucleic acid,” and “nucleic acid sequence” refer to deoxyribonucleotides or ribonucleotides and polymers thereof in either single or double stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed base and/or deoxyinosine residues (Batzer et al. (1991) Nucleic Acid Res 19:5081; Ohtsuka et al. (1985) J Biol Chem 260:2605 2608; Rossolini et al. (1994) Mol Cell Probes 8:91 98).

The terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted.

The term “isolated”, when used in the context of an isolated nucleotide or an isolated polypeptide, is a nucleotide or polypeptide that, by the hand of man, exists apart from its native environment and is therefore not a product of nature. An isolated nucleotide or polypeptide can exist in a purified form or can exist in a non-native environment such as, for example, in a transgenic host cell.

The term Del-1 polypeptide is inclusive of a full-length Del-1, as well as fragments thereof.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

A fragment of a Del-1 reference protein can be about 479, 478, 477, 476, 475, 474, 473, 472, 470, 469, 468, 467, 466, 465, 464, 463, 462, 461, or 460 amino acids long. In some embodiments, a Del-1 polypeptide that is a fragment of a Del-1 reference protein in which about 1, 2, 3, 4, or 5 amino acid residues are deleted from the amino-terminus of the full-length reference Del-1 polypeptide. In some embodiments, a Del-1 polypeptide that is a fragment of a Del-1 reference protein in which about 1, 2, 3, 4, or 5 amino acid residues are deleted from the carboxy-terminus of the full-length reference Del-1 polypeptide. In some embodiments, a Del-1 polypeptide that is a fragment of a Del-1 reference protein includes a preserved RGD motif.

In some embodiments, the Del-1 polypeptide can be a polypeptide comprising fragment of SEQ ID NO: 1. In some embodiments, the Del-1 polypeptide includes a fragment of the polypeptide of SEQ ID NO: 1, wherein the fragment is about 479, 478, 477, 476, 475, 474, 473, 472, 470, 469, 468, 467, 466, 465, 464, 463, 462, 461, or 460 amino acids long. In some embodiments, the Del-1 polypeptide includes a fragment of the polypeptide of SEQ ID NO: 1, in which about 1, 2, 3, 4, or 5 amino acid residues are deleted from the amino-terminus of the polypeptide of SEQ ID NO: 1. In some embodiments, the Del-1 polypeptide includes a fragment of the polypeptide of SEQ ID NO: 1, in which about 1, 2, 3, 4, or 5 amino acid residues are deleted from the carboxy-terminus of the polypeptide of SEQ ID NO: 1. In some embodiments, a Del-1 polypeptide includes a fragment of the polypeptide of SEQ ID NO: 1, in which the RGD motif is preserved.

In some embodiments, the Del-1 polypeptide can be a polypeptide encoded by a nucleic acid comprising a fragment of the nucleic acid of SEQ ID NO: 2. In some embodiments, the Del-1 polypeptide is encoded by a nucleic acid including a fragment of the nucleic acid of SEQ ID NO: 2, wherein the fragment is about 1437, 1436, 1435, 1434, 1433, 1432, 1431, 1430, 1429, 1428, 1427, 1426, 1425, 1424, 1423, 1422, 1421, 1420, 1419, 1418, 1417, 1416, 1415, 1414, 1413, 1412, 1411, 1410, 1409, 1408, 1407, 1406, 1405, 1404, 1403, 1402, 1401, 1400, 1399, 1398, 1397, 1396, 1395, 1394, 1393, 1392, 1391, 1390, 1389, 1388, 1387, 1386, 1385, 1384, 1383, 1382, 1381, or 1380 nucleotides long. In some embodiments, the Del-1 polypeptide is encoded by a nucleic acid including a fragment of the nucleic acid of SEQ ID NO: 2, in which about 1, 2, 3, 4, 5, 6, 7, 8, 9, 10, 11, 12, 13, 14, or 15 nucleic acid residues are deleted from the 3′ end of the nucleic acid of SEQ ID NO: 2. In some embodiments, the Del-1 polypeptide is encoded by a nucleic acid including a fragment of the nucleic acid of SEQ ID NO: 2, in which about 1, 2, 3, 4, 5, 6, 7, 8, 9, 10, 11, 12, 13, 14, or 15 nucleic acid residues are deleted from the 5′ end of the nucleic acid of SEQ ID NO: 2. In some embodiments, the Del-1 polypeptide is encoded by a nucleic acid including a fragment of the nucleic acid of SEQ ID NO: 2, where the nucleic acid residues encoding the RGD motif are preserved.

In some embodiments, the Del-1 polypeptide can be a polypeptide having about 85, 86, 87, 88, 89, 90, 91, 92, 93, 94, 95, 96, 97, 98, or 99% homology or identity to the polypeptide of SEQ ID NO: 1. In some embodiments, the Del-1 polypeptide can be a polypeptide having about 85, 86, 87, 88, 89, 90, 91, 92, 93, 94, 95, 96, 97, 98, or 99% homology or identity to a fragment of the polypeptide of SEQ ID NO: 1, wherein the fragment is about 479, 478, 477, 476, 475, 474, 473, 472, 470, 469, 468, 467, 466, 465, 464, 463, 462, 461, or 460 amino acids long. In some embodiments, the Del-1 polypeptide can be a polypeptide having about 85, 86, 87, 88, 89, 90, 91, 92, 93, 94, 95, 96, 97, 98, or 99% homology or identity to a fragment of the polypeptide of SEQ ID NO: 1, in which about 1, 2, 3, 4, or 5 amino acid residues are deleted from the carboxy-terminus of the polypeptide of SEQ ID NO: 1. In some embodiments, the Del-1 polypeptide can be a polypeptide having about 85, 86, 87, 88, 89, 90, 91, 92, 93, 94, 95, 96, 97, 98, or 99% homology or identity to a fragment of the polypeptide of SEQ ID NO: 1, in which about 1, 2, 3, 4, or 5 amino acid residues are deleted from the amino-terminus of the polypeptide of SEQ ID NO: 1. In some embodiments, the Del-1 polypeptide can be a polypeptide having about 85, 86, 87, 88, 89, 90, 91, 92, 93, 94, 95, 96, 97, 98, or 99% homology or identity to a fragment of the polypeptide of SEQ ID NO: 1, in which the RGD motif is preserved.

In some embodiments, the Del-1 polypeptide can be a polypeptide encoded by a nucleic acid having about 85, 86, 87, 88, 89, 90, 91, 92, 93, 94, 95, 96, 97, 98, or 99% homology or identity to the nucleic acid of SEQ ID NO: 2. In some embodiments, the Del-1 polypeptide can be a polypeptide encoded by a nucleic acid having about 85, 86, 87, 88, 89, 90, 91, 92, 93, 94, 95, 96, 97, 98, or 99% homology or identity to a fragment of the nucleic acid of SEQ ID NO: 2, wherein the fragment is about 1437, 1436, 1435, 1434, 1433, 1432, 1431, 1430, 1429, 1428, 1427, 1426, 1425, 1424, 1423, 1422, 1421, 1420, 1419, 1418, 1417, 1416, 1415, 1414, 1413, 1412, 1411, 1410, 1409, 1408, 1407, 1406, 1405, 1404, 1403, 1402, 1401, 1400, 1399, 1398, 1397, 1396, 1395, 1394, 1393, 1392, 1391, 1390, 1389, 1388, 1387, 1386, 1385, 1384, 1383, 1382, 1381, or 1380 nucleotides long. In some embodiments, the Del-1 polypeptide can be a polypeptide encoded by a nucleic acid having about 85, 86, 87, 88, 89, 90, 91, 92, 93, 94, 95, 96, 97, 98, or 99% homology or identity to a fragment of the nucleic acid of SEQ ID NO: 2, in which about 1, 2, 3, 4, 5, 6, 7, 8, 9, 10, 11, 12, 13, 14, or 15 nucleic acid residues are deleted from the 3′ end of the nucleic acid of SEQ ID NO: 2. In some embodiments, the Del-1 polypeptide can be a polypeptide encoded by a nucleic acid having about 85, 86, 87, 88, 89, 90, 91, 92, 93, 94, 95, 96, 97, 98, or 99% homology or identity to a fragment of the nucleic acid of SEQ ID NO: 2, in which about 1, 2, 3, 4, 5, 6, 7, 8, 9, 10, 11, 12, 13, 14, or 15 nucleic acid residues are deleted from the 5′ end of the nucleic acid of SEQ ID NO: 2. In some embodiments, the Del-1 polypeptide can be a polypeptide encoded by a nucleic acid having about 85, 86, 87, 88, 89, 90, 91, 92, 93, 94, 95, 96, 97, 98, or 99% homology or identity to a fragment of the nucleic acid of SEQ ID NO: 2, where the nucleic acid residues encoding the RGD motif are preserved.

“Percent identity,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). [BLAST nucleotide searches are performed with the NBLAST program, score+100, wordlength=12, to obtain nucleotide sequences homologous to a nucleic acid molecule of the invention. BLAST protein searches are performed with the XBLAST program, score=50, word length=3, to obtain amino acid sequences homologous to a reference polypeptide (e.g., SEQ ID NO: X). To obtain gapped alignments for comparison purposes, Gapped BLAST is utilized as described in Altschul, et al. (Nucleic Acids Res. 25: 3389-3402, 1997). When utilizing BLAST and Gapped BLAST programs, the default parameters of the respective programs (e.g., XBLAST and NBLAST) are used. See http://www.ncbi.nlm.nik.gov.

Some embodiments of the method of the presently-disclosed subject matter involve monitoring the subject before, during, and/or after receiving Del-1 polypeptide treatment. For example, in some embodiments, the subject can be monitored for periodontal inflammation and/or bone loss. As will be recognized by one of ordinary skill in the part upon study of this document, depending on the situation and/or timing of the monitoring relative to the administration of the Del-1 polypeptide, such monitoring can be useful, for example, for identifying subject having periodontitis or a risk thereof and/or for assessing the efficacy of a treatment program including administration of the Del-1 polypeptide.

In some embodiments, monitoring the subject includes monitoring the subject for periodontal inflammation and/or bone loss. In some embodiments, determinations of periodontal inflammation and/or bone loss can be made by examination of the subject by a health care professional. In some embodiments, monitoring the subject includes monitoring a marker associated with periodontal inflammation and/or bone loss. Examples of markers associated with periodontal inflammation and/or bone loss will be recognized by those of ordinary skill in the art and include, but are not limited to, IL-1β and MMP-8. Such markers can be monitored using techniques known to those of ordinary skill in the art. For example, gingival crevicular fluid (GCF) can be extracted and markers can be identified using an ELISA.

Some embodiments of the method of the presently-disclosed subject matter involve identifying a subject having periodontitis, including periodontal inflammation and/or bone loss, or a risk thereof. In some embodiments, such identification occurs prior to administration of the Del-1 polypeptide.

As noted herein, older age is associated with an increased prevalence and severity of periodontitis. As such, in some embodiments, the Del-1 polypeptide is administered to a subject who is identified as having a risk of periodontitis upon reaching a predetermined age. In some embodiments, the subject can be identified for receiving treatment in accordance with the presently-disclosed subject matter based on a pre-screening involving a consideration to the age of the subject. The age or predetermined age can be, for example, at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60.

Increased risk of periodontitis also occurs in younger subjects, who suffer from particular conditions. For example, individuals with conditions or syndromes affecting neutrophil recruitment to peripheral tissues rapidly develop severe periodontitis early in life (Waldrop (1987), Deas (2003), Nussbaum (2011)). Neutrophil dysfunction-associated periodontitis in children leads to loss of primary and permanent teeth, causing serious adverse psychological and functional consequences (esthetics, mastication, and speech).

A number of conditions prevent the normal extravasation of neutrophils and their chemotactic recruitment to sites of infection or inflammation. In leukocyte adhesion deficiency (LAD), circulating leukocytes have defects in the expression or function of integrins, or other adhesion molecules, and consequently cannot adhere to vascular endothelial cells and transmigrate to peripheral tissues. LAD represents a group of inherited disorders: LAD I is caused by deficiency in β₂ integrins, LAD II is due to defective glycosylation of selectin ligands, and LAD III involves dysfunction of signaling intermediates affecting integrin activation. The defective neutrophil adhesion caused by these conditions is replicated in mice lacking the corresponding genes. In these knockout mice, the neutrophils cannot adhere to the vascular endothelium and thus fail to migrate normally, even in the presence of appropriate chemotactic stimuli. Neutrophils from patients with Wiskott-Aldrich syndrome (or from mice deficient in the Wiskott-Aldrich protein) also display defective adhesion and transmigration, owing to impaired β₂ integrin clustering. Impaired neutrophil migration can also arise from defective chemotaxis as seen in individuals with Chediak-Higashi syndrome, lazy leukocyte syndrome, Papillon-LeFevre syndrome, or Down's syndrome. Patients with LAD or other syndromes mentioned above suffer from recurrent infections and also develop rapidly advancing inflammatory bone loss starting at very young age and leading to premature tooth loss (Waldrop (1987), Deas (2003), Nussbaum (2011), Kinane (2003), Dababneh (2008), Etzioni (2000), Sollecito (2005)). Disorders affecting the numbers of neutrophils, such as congenital agranulocytosis, cyclic neutropenia (idiopathic or drug-induced), autoimmune neutropenia, chronic idiopathic neutropenia, HIV-associated neutropenia, and neutropenia in cancer patients under chemotherapy or radiation therapy. Unless the neutrophil count is appropriately corrected, these conditions are strongly associated with severe periodontitis (Deas (2003), Nussbaum (2011), Sollecito (2005)).

As such, in some embodiments of the presently-disclosed subject matter, the Del-1 polypeptide is administered to a subject having a condition associated with neutrophil dysfunction. In some embodiments, the Del-1 polypeptide is administered to a subject having a condition selected from: a leukocyte adhesion deficiency (LAD), Wiskott-Aldrich syndrome, Chediak-Higashi syndrome, lazy leukocyte syndrome, Papillon-LeFevre syndrome, Down's syndrome, congenital agranulocytosis, cyclic neutropenia (idiopathic or drug-induced), autoimmune neutropenia, chronic idiopathic neutropenia, HIV-associated neutropenia, and neutropenia in cancer patients under chemotherapy and/or radiation therapy.

As noted hereinabove, periodontitis is a chronic inflammatory disease that constitutes a risk factor for systemic diseases, including, other chronic inflammatory conditions, adverse pregnancy outcomes, aspiration pneumonia, atherosclerosis, chronic obstructive pulmonary disease, diabetes, inflammatory bowel diseases, and rheumatoid arthritis. As such, the methods, compositions, and kits of the presently-disclosed subject matter have utility in reducing the risk of developing a chronic inflammatory condition, adverse pregnancy outcomes, aspiration pneumonia, atherosclerosis, chronic obstructive pulmonary disease, diabetes, inflammatory bowel diseases, and/or rheumatoid arthritis. Methods of the presently-disclosed subject matter include administering a Del-polypeptide for the treatment of a chronic inflammatory condition, adverse pregnancy outcomes, aspiration pneumonia, atherosclerosis, chronic obstructive pulmonary disease, diabetes, inflammatory bowel diseases, and/or rheumatoid arthritis.

As used herein, the terms “treatment” or “treating” relate to curing or substantially curing a condition, as well as ameliorating at least one symptom of the condition, and are inclusive of prophylactic treatment and therapeutic treatment. As would be recognized by one or ordinary skill in the art, treatment that is administered prior to clinical manifestation of a condition then the treatment is prophylactic (i.e., it protects the subject against developing the condition). If the treatment is administered after manifestation of the condition, the treatment is therapeutic (i.e., it is intended to diminish, ameliorate, control, or maintain the existing condition and/or side effects associated with the condition). The terms relate to medical management of a subject with the intent to substantially cure, ameliorate, stabilize, or substantially prevent a condition of interest (e.g., disease, pathological condition, or disorder), including but not limited to prophylactic treatment to preclude, avert, obviate, forestall, stop, or hinder something from happening, or reduce the severity of something happening, especially by advance action. As such, the terms treatment or treating include, but are not limited to inhibiting the progression of a condition of interest; arresting or preventing the development of a condition of interest; reducing the severity of a condition of interest; ameliorating or relieving symptoms associated with a condition of interest; causing a regression of the condition of interest or one or more of the symptoms associated with the condition of interest; and preventing a condition of interest or the development of a condition of interest. The terms includes active treatment, that is, treatment directed specifically toward the improvement of a condition of interest, and also includes causal treatment, that is, treatment directed toward removal of the cause of the condition of interest. In addition, the terms includes palliative treatment, that is, treatment designed for the relief of symptoms rather than the curing of the condition of interest; preventative treatment, that is, treatment directed to minimizing or partially or completely inhibiting the development of the associated condition of interest; and supportive treatment, that is, treatment employed to supplement another specific therapy directed toward the improvement of the associated condition of interest.

As used herein, the term “subject” refers to a target of administration. The subject of the herein disclosed methods can be a human or an animal having gingiva (gums). Thus, veterinary therapeutic uses are provided in accordance with the presently disclosed subject matter. As such, the presently disclosed subject matter provides for administration to mammals such as humans and non-human primates, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; rabbits, guinea pigs, and rodents. Also provided is the treatment of livestock, including, but not limited to, domesticated swine, ruminants, ungulates, horses (including race horses), and the like.

As used herein, the term “effective amount” refers to a dosage of a Del-1 polypeptide sufficient to provide treatment for the condition of interest being treated. This can vary depending on the subject, the condition, and the treatment program. The exact amount that is required will vary from subject to subject, depending on the species, age, and general condition of the subject, the particular carrier or adjuvant being used, mode of administration, and the like. As such, the effective amount will vary based on the particular circumstances, and an appropriate effective amount can be determined in a particular case by one of ordinary skill in the art using only routine experimentation.

In some embodiments of the presently-disclosed method, composition, and kit, the Del-1 polypeptide can be provided in a fusion protein. As will be recognized by one of ordinary skill in the art, certain benefits can be conferred by providing a therapeutic polypeptide in a fusion protein with one or more other polypeptides. In some embodiments of the presently-disclosed method, the Del-1 polypeptide can be provided as a fusion protein with IgG Fc. Without wishing to be bound by theory or mechanism, providing the Del-1 polypeptide as a fusion protein with IgG Fc can provide stability and increased bioavailability. Further, it is found that the fusion protein with IgG Fc dimerizes the Del-1 polypeptide, which can be desirable in some embodiments.

As noted herein, the presently-disclosed subject matter includes a composition comprising a Del-1 polypeptide formulated for delivery to an oral cavity of a subject. When the term Del-1 polypeptide is used in connection with methods, uses, and kit as described herein, the term is inclusive of a Del-1 polypeptide, as well as a composition comprising a Del-1 polypeptide. In some embodiments, a composition of the presently-disclosed subject matter includes a Del-1 polypeptide and a vehicle, appropriate for delivery to an oral cavity of a subject, in which the polypeptide is provided. As noted herein, the Del-1 polypeptide can be provided as a fusion protein. As such, compositions of the presently-disclosed subject matter can include a Del-1 polypeptide provided as a fusion protein and formulated for delivery to an oral cavity of a subject.

As used herein, the terms “administering” and “administration” refer to any method of delivering a Del-1 polypeptide to a subject to affect treatment. Such methods are well known to those skilled in the art and include, but are not limited to, administration to the gingiva or periodontal pocket, e.g., by topical application, microinjection, insertion into the pocket. In some embodiments, the Del-1 polypeptide is administered to the mouth of the subject. In some embodiments, the Del-1 polypeptide is administered to a gingiva of the subject. In some embodiments, the Del-1 polypeptide is administered to a periodontal pocket of the subject. Without wishing to be bound by theory or mechanism, it is noted that a Del-1 polypeptide can be applied locally to affect treatment of periodontal inflammation and/or bone loss, such that there is not any interference with immunity response (Del-1 polypeptide contemplated to have ability to limit leukocyte recruitment to sites of inflammation to impair the ability of subject to fight infection).

Various administration protocols can be used in connection with the presently-disclosed subject matter. In some embodiments, the Del-1 polypeptide can be administered multiple times. In some embodiments, the Del-1 polypeptide can be administered periodically. For example, the Del-1 polypeptide can be administered at least 4 times daily, 3 times daily, 2 times daily, daily, every other day, every third day, every fourth day, every fifth day, every sixth day, or weekly. In some embodiments, the Del-1 polypeptide can be administered less-frequently, particularly when at higher doses and/or when provided in a slow-release formulation. In some embodiments, the Del-1 polypeptide can be administered periodically for 1, 2, 3, 4, 5, 6, 7, 8, 9, 10, 11, or 12 months. In some embodiments, the Del-1 polypeptide can be administered periodically for an indefinite period, as part of a dental hygiene regimen, e.g., when brushing teeth, flossing teeth, using mouthwash, etc. In some embodiments, the Del-1 polypeptide can be administered in multiple doses. A dose can include a discrete amount of the Del-1 polypeptide useful for a single administration. In some embodiments, the Del-1 polypeptide

As will be recognized by the skilled artisan, the administration, including most effective manner and/or protocol for administration, of the Del-1 polypeptide can vary depending on the manner in which the Del-1 polypeptide is formulated. The Del-1 polypeptide can be provided in any appropriate form for delivery to the oral cavity of a subject that is known to one of ordinary skill in the art. An appropriate vehicle will be by bioacceptable (appropriate vehicles will not result in unacceptable toxicity when delivered to the oral cavity of a subject). The following description is merely exemplary of forms in which the Del-1 polypeptide can be provided. In some embodiments of the presently-disclosed subject matter the Del-1 polypeptide is provided in a delivery vehicle selected from: a toothpaste, a mouthwash, a chewing gum, a dental floss, a beverage, a food product, a gel, including a slow-release gel, a tablet, a granule, and a film, including a thin film of biodegradable matrix. As such, for example, an effective manner and protocol for administration of a Del-1 polypeptide formulated in a toothpaste, a mouthwash, or a dental floss can be to use the toothpaste, mouthwash and/or dental floss in connection with a typical or recommended dental hygiene regimen. For another example, a Del-1 polypeptide could be formulated in a chewing gum, provided in a pack containing a convenient and/or recommended number of doses (each provided in a discrete piece of chewing gum) for use as a chewing gum during a treatment period. For another example, a Del-1 polypeptide could be formulated in a gel for application to the gingiva/into the periodontal pocket using a device, such as a small syringe or stick. For another example, a Del-1 polypeptide could be formulated in a thin film of biodegradable matrix can be inserted into the periodontal pocket, as will be recognized by one or ordinary skill in the art.

In some embodiments of the presently-disclosed subject matter the Del-1 polypeptide can be expressed by a cell comprising a nucleotide encoding the Del-1 polypeptide, which nucleic acids encoding Del-1 polypeptides are disclosed hereinabove. The cell expressing the Del-1 polypeptide can be transfected with a vector comprising a nucleic acid encoding the Del-1 polypeptide. In some embodiments, the Del-1 polypeptide can be expressed in the chloroplasts of a plant. A subject can receive the Del-1 polypeptide by chewing the leaves of the plant. In some embodiments, the Del-1 polypeptide can be expressed in a bacteria, which is administered as probiotics are administered, e.g., in granules, yogurt, etc.

The presently-disclosed subject matter further includes a kit. In some embodiments, the kit includes a Del-1 polypeptide and a device for administering the Del-1 polypeptide or composition. Appropriate devices will be recognized by one or ordinary skill in the art, but can include, for example, a syringe or a toothbrush. In some embodiments, a kit can include multiple doses of the Del-1 polypeptide. In some embodiments, the multiple doses are included in a packet of chewing gum.

While the terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

### Introduction to Examples

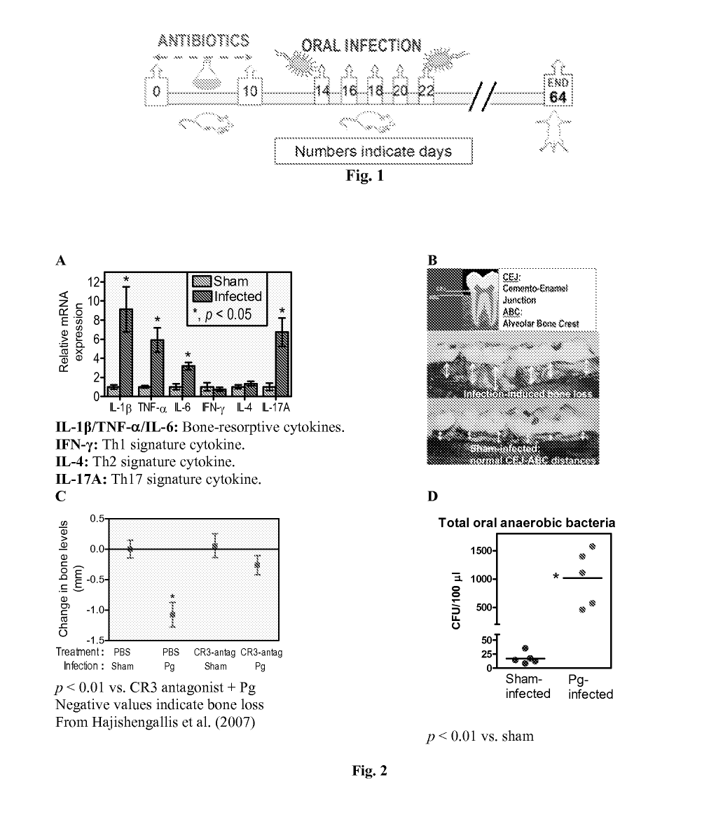

Developmental endothelial locus-1 (Del-1), also known as EGF-like repeats and discoidin I-like domains 3 (EDIL3) is an endothelial cell-expressed 52-kDa glycoprotein, originally described for its role in vascularization (Penta (1999)). Although secreted by endothelial cells, Del-1 can become associated with the endothelial cell surface by binding to surface proteoglycans or to integrin αvβ3 by means of an Arg-Gly-Asp (RGD) motif on the second EGF-like repeat (Chavakis (2009), Hidai (2007)). More recently, Del-1 was shown to be a novel ligand for the LFA-1 integrin but, unlike ICAM-1, it antagonizes LFA-1-dependent leukocyte adhesion onto the vascular endothelium (FIG. 1 of Choi (2008)). Importantly, at equimolar amounts, Del-1 outcompetes ICAM-1 for binding to LFA-1 on leukocytes and thereby inhibits the adhesion and diapedesis of neutrophils (Choi (2008)). These findings suggest that Del-1 acts homeostatically to regulate local inflammation. As described in the Examples below, this concept was demonstrated in an animal model of periodontitis, a chronic inflammatory disease, which leads to the destruction of the tissues that surround and support the teeth (periodontium) and constitutes a risk factor for systemic diseases (e.g., atherosclerosis, rheumatoid arthritis, adverse pregnancy outcomes, diabetes, and aspiration pneumonia) (Pihlstrom (2005), Genco (2010), Lalla (2011), Tonetti (2007), Jeffcoat (2011), Lundberg (2010)).

The role of Del-1 in periodontal homeostasis was established in distinct models. Del-1 expression is diminished in gingival tissue in old age, correlating with excessive neutrophil recruitment and IL-17-dependent inflammatory bone loss, the hallmark of periodontitis, as described in the Examples below. Consistent with this, Del-1-deficient mice develop spontaneous inflammatory bone loss at young age accompanied by heavy neutrophil infiltration in the gingiva, and inflammation is dependent on LFA-1 and the IL-17 receptor. Whereas Del-1 inhibits LFA-1-dependent neutrophil recruitment and IL-17 production, IL-17 downregulates Del-1 expression in endothelial cells and thereby promotes neutrophil recruitment. Therefore, Del-1 and IL-17 are reciprocally cross-regulated and, moreover, the inhibition of Del-1 by IL-17 is a novel mechanism by which IL-17 can facilitate neutrophil recruitment to sites of inflammation. Other, previously established mechanisms include the capacities of IL-17 to orchestrate granulopoiesis and neutrophil mobilization and chemotaxis (Kolls (2004), Stark (2005), Gaffen (2009b)). The inverse expression of Del-1 and IL-17 is also observed in human gingival biopsy samples, with Del-1 dominating in healthy gingiva and IL-17 in inflamed gingiva. Moreover, human Del-1 inhibits LFA-1-dependent transendothelial migration of human neutrophils in vitro.

These findings, as described in the Examples below, show that Del-1 serves as a mechanism whereby a tissue can locally self-regulate persistent inflammation associated with chronic recruitment of neutrophils. Further, local periodontal treatment with soluble Del-1 in old mice inhibits IL-17 production, LFA-1-dependent neutrophil infiltration, and bone loss.

### Example 1

Elderly individuals display increased susceptibility to chronic inflammatory diseases (Dorshkind (2009), Gomez (2008), (Pelletier (2009)). Although the underlying mechanisms are poorly understood, age-related alterations in innate immunity may cause dysregulation of the host response leading to a heightened chronic inflammatory status (Franceschi (2000), Gomez (2008), Hajishengallis (2010)). In addition to a number of other conditions, such as atherosclerosis and Alzheimer's disease, old age is also associated with increased prevalence and severity of periodontitis, an oral inflammatory disease that leads to destruction of the tooth-supporting tissues (Hajishengallis (2010), Seymour (2007)). An estimated 10-15% of the adult population develops severe periodontitis, which is a risk factor for systemic conditions, such as atherosclerosis, aspiration pneumonia, and diabetes (Seymour (2007), van der Velden (1991)). Conventional periodontal treatment is often not sufficient to control destructive inflammation and many patients develop recurrent disease for reasons that are not clear (Amritage (2000))(2). Described in this example is a mechanism linking advanced age to destructive periodontal inflammation, which the present inventors have targeted for new therapeutic intervention against periodontitis.

Neutrophils have been implicated in the pathogenesis of several chronic inflammatory diseases including periodontitis (Kanazawa (2007), Kasama (2005), Tonetti (2007), Wang (2007)). Neutrophils can mediate tissue destruction directly (Wang (2007)), but are also involved in chemotactic recruitment of Th17 cells (Pihlstrom (2005)), which can cause chronic bone immunopathology mainly through the production of interleukin-(IL)-17 (also known as IL-17A) (Niederman (2001)). As described herein, the present inventors propose that old age is associated with dysregulated, excessive neutrophil recruitment to the periodontium, owing to diminished expression of an endothelial cell-secreted glycoprotein, known as developmental endothelial locus-1 (Del-1). Del-1 was recently identified as the first endogenous negative regulator of neutrophil migration to peripheral tissues by blocking the leukocyte-associated antigen-1 (LFA-1) (Choi (2008)). Although Del-1 displays a selective tissue expression pattern (Choi (2008)), studies indicate that it is also expressed in the gingivae. Importantly, the present inventors have shown that Del-1 expression is diminished in the gingiva of old mice relative to their young counterparts, resulting in excessive neutrophil recruitment to the periodontium after oral infection with periodontal bacteria. Moreover, Del-1-deficient (Del-1−/−) mice develop more inflammation and periodontal bone loss than wild-type controls.

The present inventors contemplate use of purified Del-1 (e.g., expressed as a chimeric protein with the IgG Fc fragment; Del-1-Fc) to correct the age-associated Del-1 deficiency and thereby reverse destructive periodontal inflammation. This rational therapeutic intervention is based on the concept that age-associated reduced Del-1 expression leads to excessive neutrophil influx to periodontal tissues, accompanied by increased recruitment of Th17-like and IL-17A-producing cells, resulting in excessive periodontal inflammation and bone loss. At a first stage, interventional studies necessitate the use of appropriate preclinical animal models, since they cannot normally be performed directly in humans due to important ethical considerations (Graves (2008)).

The present inventors show that treatment of old mice with Del-1-Fc reverses excessive neutrophil recruitment to periodontal tissues and leads to reduced inflammation and Th17-like activity; show that treatment with Del-1-Fc inhibits periodontal disease activity in old mice; and compare Del-1-Fc with LFA-1 and IL-17A antagonists for therapeutic treatment of periodontitis.

The experimental design is outlined herein and is based on the recently developed aging model of periodontitis (Lubberts (2008)). The studies are designed to not only establish the protective potential of Del-1 and of antagonists of downstream inflammatory molecules, but to also obtain insight on the mechanisms of action (by including experiments with single or double knock-out mice lacking combinations of the molecules of interest; Del-1, LFA-1, and IL-17 receptor).

To the best of the present inventors' knowledge this is the first time that an age-associated deficiency in periodontal innate immune regulation has been identified and a promising therapeutic intervention has been proposed.

It is contemplated that the therapeutic exploitation of Del-1 will effectively complement clinical treatment and revolutionize the way elderly periodontal patients are managed. Moreover, the same molecule could be applied to reverse other chronic inflammatory conditions, where neutrophils have been implicated in their pathogenesis, such as rheumatoid arthritis, inflammatory bowel diseases, and chronic obstructive pulmonary disease.

The present inventors discovered a basic mechanism linking advanced age to destructive periodontal inflammation, and propose a novel therapeutic intervention against periodontitis. Specifically, based on data described herein, the present inventors have shown that the expression of Del-1, a molecule that homeostatically regulates neutrophil extravasation (Choi (2008)), is reduced in old age and Del-1 deficiency is associated with excessive neutrophil recruitment to the periodontium and increased inflammation and periodontal bone loss. Without wishing to be bound by theory, it is believed that the discovery could account for the fact that the elderly show an inappropriately high periodontal inflammatory response, relative to young individuals (Fransson (1996). Specifically, in an experimental gingivitis study involving young (20-25 years of age) and elderly (≧65 years) individuals, the elderly group developed more severe gingival inflammation associated with elevated numbers of neutrophils, even though both groups displayed comparable dental plaque biofilm accumulation (Fransson (1996)). This hallmark study provided clinical evidence that aging influences the periodontal innate immune system in a way that promotes inflammation, although the underlying mechanisms remained elusive.

This age-dependent heightened chronic inflammatory status is not restricted to the periodontal tissues but is generally associated with old age and has been aptly coined as “inflamm-aging” (Franceschi (2000)). In this regard, it is possible that Del-1-based treatments could also be applied in a variety of inflammatory diseases of the elderly, especially those in which neutrophils have been implicated in their pathogenesis.

By necessity, novel therapeutic interventions require preclinical testing in appropriate animal models, for rapid, cost-effective identification of promising therapeutic compounds since, and also because such studies cannot normally be addressed directly in humans, owing to important ethical considerations (Graves (2008)). However, the approach to supplementing Del-1 to reverse a deficiency with the same molecule should be a safe approach. Therefore, establishing utility and efficacy of Del-1 to prevent or mitigate periodontitis in the preclinical model, will lead to a rapid translation to clinical management of periodontal and other inflammatory diseases. Del-1-based therapy has the potential to revolutionize the way elderly periodontal patients are managed and can lead to a substantial decrease in morbidity.

Preclinical models of periodontitis and therapeutic intervention.

A mouse model for inflammatory periodontal bone loss has been utilized in for investigating periodontal host-pathogen interactions and effectiveness of therapeutic interventions (Hajishengallis (2007), Armitage (2002)) (FIG. 1). The technical procedures have been published (Armitage (2002)) and typical investigated parameters include induction of proinflammatory mediators in the periodontium (FIG. 2A) and induction of periodontal bone loss (FIG. 2B,C). Although the model is based on oral inoculation of mice with P. gingivalis, it should be stated that, most likely, this does not represent a monoinfection. This is because P. gingivalis causes a major increase in the numbers of the indigenous oral anaerobic flora (FIG. 2D) which could thereby contribute to inflammation and bone loss. This observation is consistent with the concept of P. gingivalis constituting a keystone species in periodontitis, being capable to promote the survival and virulence of the whole biofilm community (Darveau (2010), Hajishengallis (2009)).

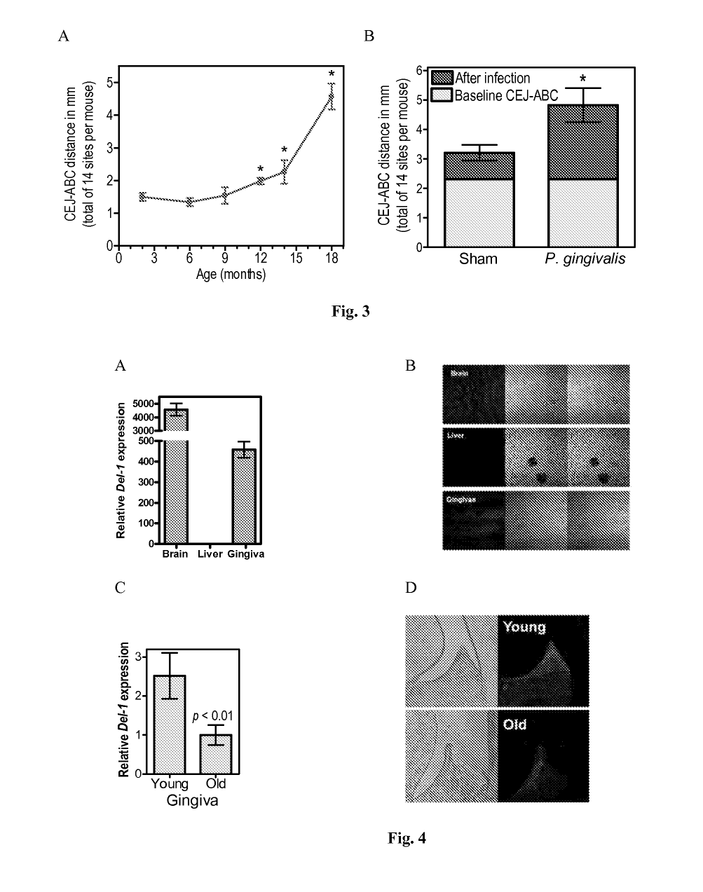

Mice used for experimental periodontitis are usually 8-12 week-old and sham-infected mice do not typically develop appreciable periodontal bone loss. However, mice with genetically altered immune status (e.g., IL-10 deficiency) spontaneously develop periodontal bone loss (i.e., even when they are not inoculated with human pathogens) that can be reversed by antibiotic treatment (Al-Rasheed (2003), Pawelec (2008)). This suggested that the indigenous oral microbiota of mice contains potential periodontal pathogens and, therefore, mice may develop periodontitis in advanced age like humans. Indeed, the present inventors have shown that mice develop age-dependent inflammatory periodontal bone loss which becomes quite dramatic after 9 months of age (Lubberts (2008)) (FIG. 3A). However, oral infection of old mice with P. gingivalis accelerates the age-dependent induction of periodontal bone loss (FIG. 3B). The age-associated increase of periodontal bone loss is accompanied by elevated expression of proinflammatory cytokines (IL-1β, TNF-α, and IL-17A) (Lubberts (2008)). Although IL-1β and TNF-α are major mediators of destructive bone resorption in periodontitis, the role of IL-17A in periodontitis has not been established (Gaffen (2008), Graves (2008)). However, IL-17A is the major effector cytokine of the Th17 lineage, which has emerged as a specialized osteoclastogenic T cell subset with a prominent role in chronic inflammatory or autoimmune diseases (Gaffen (2008)).

Del-1 is expressed in the gingiva and expression is reduced with age.

The recent identification of a soluble endothelial-derived glycoprotein, termed Del-1, as the first indigenous negative regulator of leukocyte migration to sites of inflammation (Choi (2008)) has been a pivotal advancement in the field of innate immunity. Moreover, the present inventors' own work has placed this seminal discovery in the context of “inflamm-aging”, i.e., the heightened chronic inflammatory status associated with old age. Del-1 was reported to display a selective expression pattern, since it is expressed in the brain and lung, but not in the liver, spleen, or whole blood (Choi (2008)). A search on a microarray database of age-dependent gene expression in the mouse gingiva has revealed that Del-1 is also expressed in the gingivae and, strikingly, is among the top 5% most differentially regulated genes as a function of age (not shown). To confirm and strengthen the microarray data using a more quantitative approach, the present inventors performed real-time PCR to determine Del-1 gene expression levels, followed by immunohistochemistry to confirm the findings at the protein level. In the first experiment, young C57BL/6 mice (8-10 weeks old) were sacrificed and their gingival tissues, brains and livers were collected into RNA later (Ambion) (for quantitative real-time PCR [qPCR]) or into 4% paraformaldehyde (for immunohistochemistry). The present inventors found that Del-1 mRNA was expressed in the gingiva and brain (positive control) but not in the liver (negative control) (FIG. 4A). Moreover, the present inventors could readily detect Del-1 protein expression in gingiva and brain but not in liver (FIG. 4B). The present inventors then investigated if Del-1 expression is indeed reduced by age. If true, this could be a major mechanism accounting for enhanced influx of neutrophils and therefore increased inflammation in old age. Young (8-10 weeks old) and old (≧18 months) C57BL/6 mice (both age groups obtained from the National Institute of Aging) were used in this experiment and gingival tissues were used as above. The present inventors found that gingiva harvested from young mice displayed 2.5 times higher expression of Del-1 mRNA than periodontal tissues from old mice (FIG. 4C), although markedly more pronounced differences were noted at the protein level (FIG. 4D). This age-associated reduction in Del-1 expression suggested a possible dysregulation of neutrophil recruitment in periodontitis, and could be an explanation as to why the elderly show an inappropriately increased inflammatory response to oral pathogens.

Age-associated reduced Del-1 expression correlates with increased neutrophil recruitment to the periodontium.

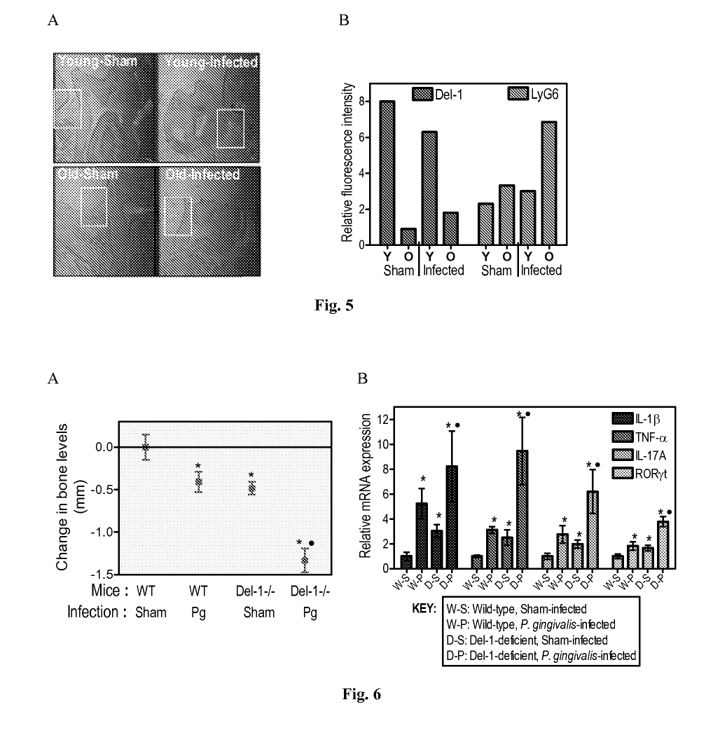

The present inventors then investigated if reduced Del-1 expression in periodontal tissues of old mice correlates with increased neutrophil accumulation (relative to young controls) in response to P. gingivalis challenge. For this purpose, the present inventors infected young (8-10 weeks old) and old (≧18 months) C57BL/6 mice with P. gingivalis or vehicle only (2% carboxymethylcellulose/PBS; Sham) for 24 hours. Harvested periodontal tissues were processed and fluorescently stained using antibodies specific for Del-1 and LyG6 (a specific neutrophil marker). Del-1 expression was lower in old mice, both under sham- or P. gingivalis-infected conditions, whereas higher numbers of neutrophils were seen in the periodontal tissue of old mice upon P. gingivalis infection, compared to their young counterparts (FIG. 5). Thus, age-associated deficiency in Del-1 appears to predispose to increased periodontal inflammation. To determine whether Del-1 deficiency can cause or facilitate periodontitis, the present inventors used the mouse periodontitis model (FIG. 1) employing Del-14-mice and normal controls.

Del-1 deficiency leads to increased periodontal disease activity.

Since Del-1 may homeostatically regulate neutrophil extravasation and ensuing inflammation (Choi (2008)), the present inventors hypothesized that the ability of P. gingivalis to cause inflammatory periodontal bone loss in mice will be enhanced in the background of Del-1 deficiency. This hypothesis was verified since P. gingivalis-infected Del-14-mice exhibited dramatically higher bone loss than similarly infected wild-type mice (FIG. 6A). Moreover, strikingly, sham-infected Del-1−/− mice exhibited significant bone loss, which was higher than sham-infected wild-type mice and comparable to P. gingivalis-infected wild-type mice (FIG. 6A). These findings suggest that Del-1 deficiency not only leads to higher susceptibility to P. gingivalis-induced periodontitis but it may also promote naturally occurring periodontitis, perhaps by promoting inflammatory responses to indigenous oral bacteria. In this regard, not only P. gingivalis-infected groups but also Del-1-deficient sham-infected mice displayed higher induction of established proinflammatory bone-resorptive cytokines, e.g., IL-1β and TNF-α (FIG. 6B). Quite intriguingly, all groups showing periodontal bone loss, and especially the P. gingivalis-infected Del-1−/− mice, exhibited increased mRNA expression for IL-17A and RORγt, a transcription factor required for Th17 cell differentiation (Gaffen (2008)). This suggests the presence of IL-17A-producing cells, very likely Th17 cells, which appear to be chemoattracted to sites of inflammation through crosstalk with neutrophils (Pihlstrom (2005)).

### Example 2

### Local Administration of Del-1 or Anti-IL-17 Inhibits Bone Loss in 18-Month Old Mice

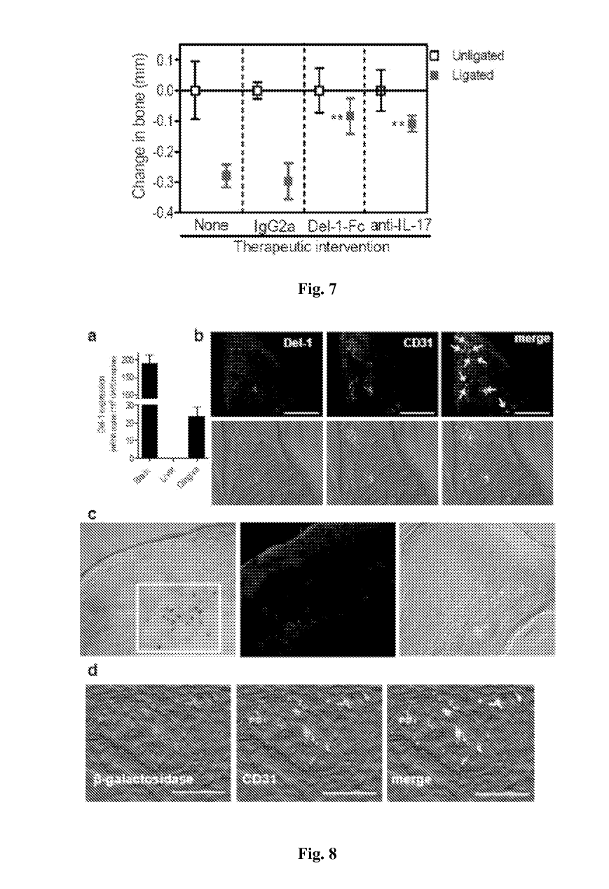

The present inventors investigated whether Del-1 could additionally inhibit induction of bone loss. In order to induce rapid bone loss (which would facilitate short-term Del-1 intervention compared to its long-term delivery in mice until old age), the ‘ligature model’ was used, where a silk ligature is placed around the second maxillary molar resulting in massive bacterial accumulation and induction of bone loss in conventional but not germ-free rodents. With reference to FIG. 7, Del-1 expressed as an Fc fusion protein, which may increase its bioavailability in the tissue, was microinjected in the palatal gingiva of the ligated second molar, one day before placement of the ligature and every day thereafter until the day before sacrifice (day 5), leading to significant inhibition of bone loss (p<0.01). In a parallel experiment utilizing the same protocol, treatment with a neutralizing monoclonal antibody (mAb) to IL-17 also resulted in significant inhibition of bone loss. In contrast, treatment with IgG2a (Fc control and isotype control for the anti-IL-17 mAb) was without any effect. These data provide proof-of-concept evidence that Del-1 has therapeutic potential for the treatment of periodontal inflammation and bone loss, and perhaps other diseases involving bone immunopathology. Data are means+/−SD (n=5 mice per group); negative values indicate bone loss.

### Example 3

Circulating neutrophils readily migrate to sites of extravascular infection or inflammation to control pathogenic insults. Because neutrophils display a large array of microbicidal and proinflammatory mechanisms that are potentially harmful to the host, their activation and trafficking is tightly regulated (Ley (2007), Luster (2005), (Chavakis (2009)). The extravasation of neutrophils depends on a well-coordinated adhesive cascade, including interactions of P2 integrins, such as LFA-1, with endothelial counter-receptors, such as intercellular adhesion molecules (ICAM) (Ley (2007), Luster (2005), (Chavakis (2009)). In contrast to multiple factors promoting leukocyte extravasation, little is known about endogenous inhibitors of the leukocyte adhesion cascade. In this context, a 52-kDa glycoprotein was identified, termed developmental endothelial locus-1 (Del-1), as a novel negative regulator of neutrophil extravasation that antagonizes β2-integrin-dependent adhesion onto the vascular endothelium (Choi (2008)). Pentraxin-3 is another recently identified endogenous inhibitor of neutrophil extravasation that suppresses selectin-dependent rolling (Deban (2010)). In contrast to pentraxin-3, Del-1 (also known as EGF-like repeats and discoidin I-like domains 3; encoded by Edil3) is produced by the tissue rather than the inflammatory cell itself (Choi (2008)). Specifically, Del-1 is secreted by endothelial cells and may associate with the endothelial cell surface and the extracellular matrix (Chavakis (2009), Hidai (2007)), predicting that Del-1 could regulate the local chronic inflammatory responses in tissues expressing it; however this hypothesis has not been addressed so far.

It was contemplated by the present inventors that Del-1 may serve a mechanism whereby a tissue may locally self-regulate persistent inflammation associated with chronic recruitment of neutrophils. Neutrophils are critically involved in the pathogenesis of periodontitis Nussbaum (2011), Serhan (2008)), a chronic inflammatory disease of the tooth-supporting tissues (periodontium) (Pihlstrom (2005)). Periodontitis, moreover, exerts a major impact on systemic health, as it increases the patients' risk for atherosclerosis, diabetes, chronic obstructive pulmonary disease and possibly rheumatoid arthritis (Pihlstrom (2005), Genco (2010), Tonetti (2007), Lundberg (2010), Lalla (2011)). Therefore, periodontitis represents an attractive model to determine the role of Del-1 in neutrophil-mediated chronic inflammation with impact on systemic diseases. Old age and age-associated inflammation are factors that contribute to increased prevalence and severity of periodontitis in humans and mice (Hajishengallis (2010), Huttner (2009)). Intriguingly, the analysis of periodontal tissue in young and aged mice revealed that Del-1 expression was diminished in old age, thereby correlating with inflammatory bone loss, the hallmark of periodontitis. Moreover, it was demonstrated a direct role for Del-1 expression in the periodontium in preventing local inflammatory pathology through inhibition of LFA-1 integrin-dependent neutrophil recruitment and interleukin 17 (IL-17)-mediated inflammation. It was also shown that Del-1 and IL-17 are reciprocally cross-regulated and that local administration of Del-1 down-regulates IL-17 and inhibits periodontal bone loss in an LFA-1-dependent manner. These data reveal a potentially novel approach to the treatment of periodontitis and other chronic inflammatory diseases.

Results

Old Mice Show Decreased Del-1 Expression

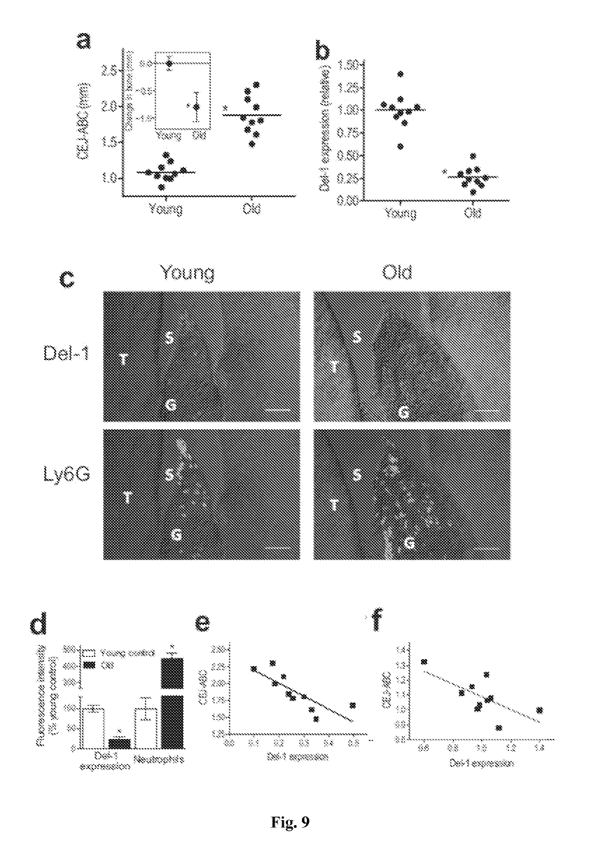

Old age is associated with increased susceptibility to inflammatory diseases (Hajishengallis (2010), Cevenini (2010), Gomez (2008)) several of which, including periodontitis, involve neutrophil-mediated tissue injury (Luster (2005), Nussbaum (2011), Serhan (2008), Cascao (2010)). Aging mice, like aging humans, can develop periodontitis (Hajishengallis (2010), Liang (2010)). It was examined whether age-related mouse periodontitis is associated with changes in Del-1 expression. Given that Del-1 is expressed in select tissues (in brain and lungs but not liver or spleen)3, the present inventors first showed that Del-1 mRNA and protein are expressed in the gingival tissue of the periodontium (FIG. 8a,b) Immunohistochemical analysis for Del-1 and Edil3-promoter driven reporter gene expression in Del-1-LacZ knockin mice revealed that Del-1 was produced locally by endothelial cells, although it could also be found in gingival extravascular areas (FIG. 8), apparently due to diffusion after its secretion by the endothelium. Similar to BALB/c mice (Liang (2010)), C57BL/6 mice developed periodontal bone loss in old age (FIG. 9a inset). Intriguingly, gingival tissue harvested from 18-month-old mice displayed about one-fourth the amount of Del-1 mRNA than gingiva from young mice (8- to 10-week old) (FIG. 9b) and a pronounced difference was also noted at the protein level (FIG. 9c, top). Interestingly, the reduced expression of Del-1 in the gingiva of old mice was associated with higher neutrophil infiltration relative to young mice (FIG. 9c,d).

The relative bone loss in old mice was calculated by measuring distances between the cementoenamel junction (CEJ) and the alveolar bone crest (ABC) (FIG. 9a inset). Linear-regression analysis of the CEJ-ABC values versus Del-1 expression (data from FIG. 9a,b, respectively) revealed a significant inverse association between Del-1 expression and periodontal bone loss in old mice (r2=0.6254, P=0.0065; FIG. 9e). This association was also significant, but not as strong, within the young group (r2=0.4641, P=0.0301; FIG. 9f). Thus, an inverse relationship between Del-1 expression and bone loss exists not only between young and old mice (FIG. 9a,b), but also within the individual age groups. These data suggest that aging is associated with periodontal Del-1 deficiency which may contribute to dysregulated or elevated neutrophil recruitment and bone loss.

Edil3−/− Mice Exhibit Enhanced Inflammation and Bone Loss

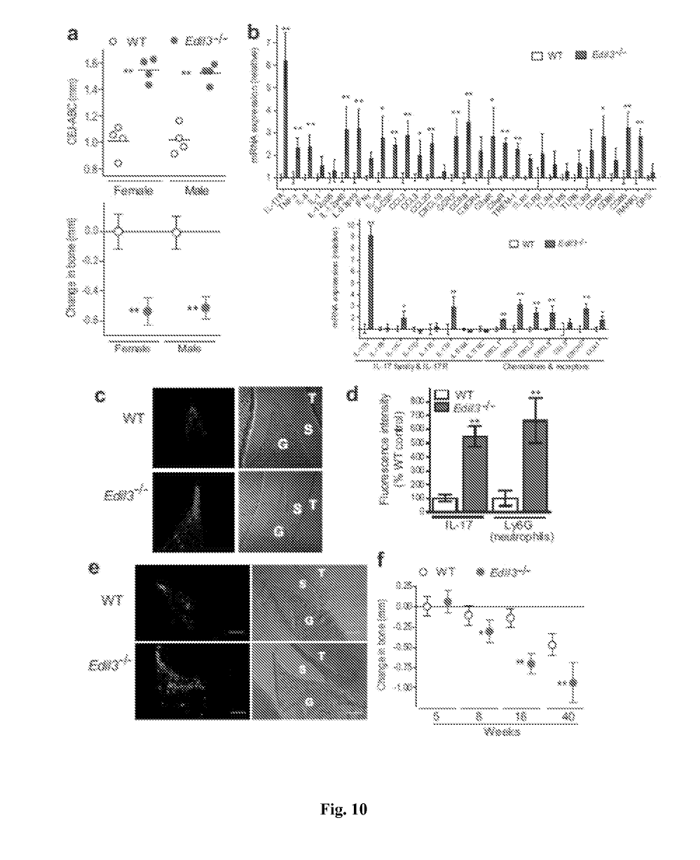

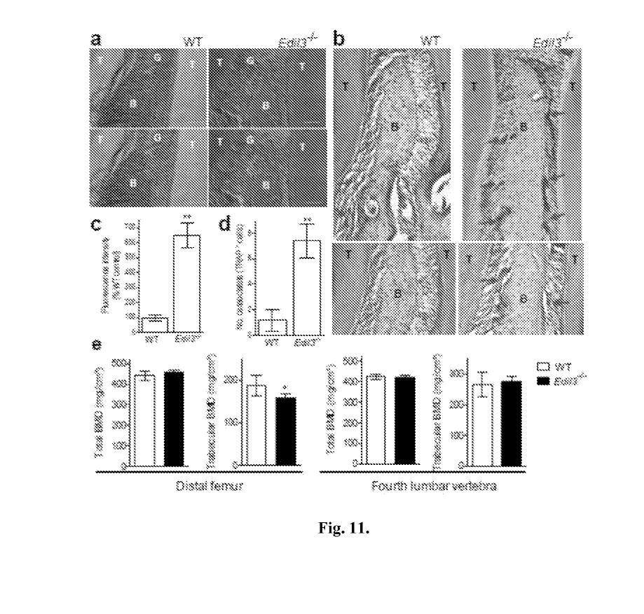

To determine a direct role for Del-1 in local inflammatory pathology, the periodontal phenotype of Edil3−/− mice was investigated. Sixteen-week-old Edil3−/− mice of either gender exhibited significant periodontal bone loss relative to their respective age-matched wild-type littermate controls (FIG. 10a). Analysis of the periodontal inflammatory response by real-time quantitative PCR (qPCR) showed significant differences between Edil3−/− mice and wild-type controls, characterized by >6-fold elevated expression of IL-17 (also known as IL-17A) upon Del-1 deficiency (FIG. 10b top). Significant but less pronounced upregulation was observed in the transcript abundance of other inflammatory molecules, such as bone-resorptive mediators (TNF, IL-6, RANKL), chemokines (CCL2, CCL20), chemokine receptors (CCR2, CCR6), receptors that amplify inflammation (C3aR, C5aR, TREM-1), and costimulatory molecules (CD40, CD86); however, both groups had comparable expression of the RANKL inhibitor, osteoprotegerin (OPG) (FIG. 10b top). Moreover, Edil3−/− mice exhibited higher gingival expression of both the p40 and p19 subunits of IL-23 (FIG. 10b top), a potent inducer of IL-17 production by both adaptive and innate immune cells (Cua (2010)).

The high expression of IL-17A and increased neutrophil infiltration in Del-1deficiency prompted us to examine possible differential expression of additional IL-17 family cytokines and neutrophil-related chemokines and receptors. IL-17F and C (but not B, D, or E) were upregulated in Del-1 deficiency, although their expression was at least one-third that of IL-17A (FIG. 10b bottom). The expression of IL-17RA and IL-17RC (the receptor subunits that recognize IL-17A and F [IL-17R] (Gaffen (2009))) was only slightly affected (FIG. 10b bottom). In comparison to wild-type controls, Edil3−/− mice expressed significantly higher amounts of CXCL-1, 2, 3, and 5 and their receptor (CXCR2); however, CCL3 expression was not affected although its receptor (CCR1) was modestly upregulated (FIG. 10b bottom). Therefore, Del-1 deficiency upregulates the expression of IL-17 cytokines (primarily the A isoform), neutrophil-recruiting CXC chemokines and their receptor, as well as the neutrophil-mobilizing agent G-CSF (FIG. 10b).

The increased expression of IL-17A protein in the Edil3−/− periodontium was confirmed by immunohistochemistry (FIG. 10c,d). Similarly, the elevated expression of RANKL protein in Del-1 deficiency was confirmed by immunohistochemistry and was accompanied by increased osteoclastic activity in the periodontium (FIG. 11a-d). Importantly, the increased inflammatory bone loss in Edil3−/− mice was associated with increased infiltration of Ly6G+ neutrophils in the gingiva (FIG. 10d,e), consistent with lack of Del-1-mediated regulation of neutrophil trafficking.

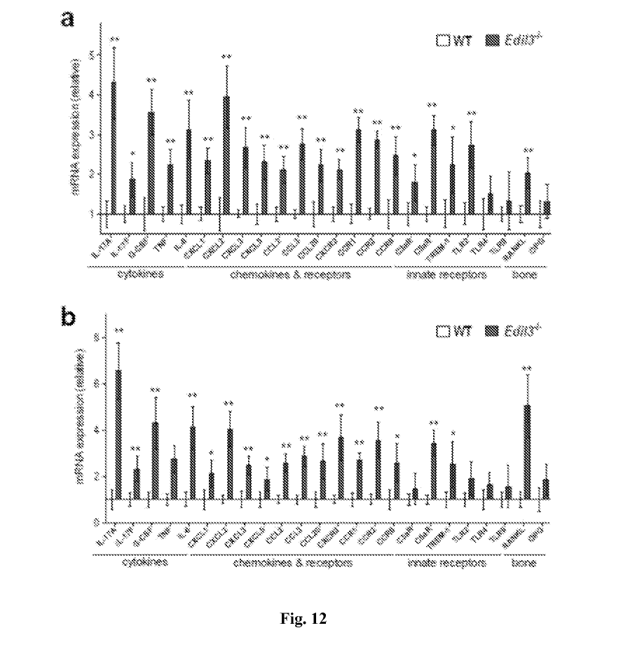

To exclude the possibility that the observed bone loss in 16-week-old Edil3−/− mice, relative to age-matched wild-type controls, was due to innately different periodontal bone heights, Edil3−/− and wild-type mice were examined at different ages. At the age of 5 weeks, no difference in the bone heights between Edil3−/− and wild-type mice was observed, whereas progressive differences were seen from the age of 8 weeks onward (FIG. 10f). Quantitative analysis of gingival mRNA expression of inflammatory mediators in Edil3−/− mice and wild-type littermate controls, at the age of 8 weeks or 9 months, revealed a similar upregulation of IL-17, neutrophil-recruiting CXC chemokines, and bone-resorptive molecules in Del-1 deficiency (FIG. 12), as seen earlier in 16-week-old mice (FIG. 10b). Therefore, the bone loss exhibited by Edil3−/− mice is an acquired rather than an innate trait and is inflammatory in nature. The impact of Del-1 deficiency on periodontal bone loss could not additionally be related to general bone defects in Edil3−/− mice, since their total bone densities in the femur and spine were comparable to that of normal mice, with only a marginal reduction in the trabecular bone density of the femur but not of the spine due to Del-1 deficiency (FIG. 11e). As female and male Edil3−/− mice had similar susceptibility to periodontitis (FIG. 10a), further studies utilized exclusively females. To allow data comparison across figures, all subsequent bone loss was calculated relative to a common baseline, determined by the bone heights of 5-week-old mice.

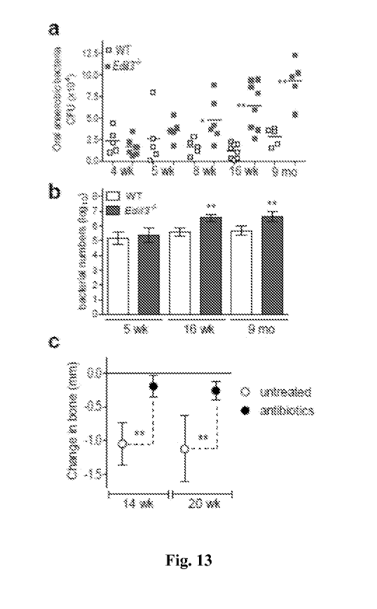

Although it is the host inflammatory response that primarily can inflict damage upon the periodontal tissue, oral anaerobic bacteria are involved in the initiation and progression of periodontitis (Gaffen (2008), Darveau (2010)). In this regard, at ≦5 weeks of age, Edil3−/− mice and wild-type littermate controls harbored comparable numbers of bacteria (determined by anaerobic culture or by qPCR of the 16S rRNA gene which would additionally enumerate unculturable bacteria); however, from the age of 8 weeks onward, Edil3−/− mice exhibited a significantly higher bacterial burden relative to age-matched wild-type controls (FIG. 13a,b). The development of periodontal bone loss in Edil3−/− mice was prevented by oral antibiotic treatment (FIG. 13c), confirming the role of bacteria in this bone loss model, as in human periodontitis (Darveau (2010)). In summary, the periodontium of Edil3−/− mice displays unregulated neutrophil infiltration, elevated bacterial burden, and inflammatory bone loss, indicating that Del-1 deficiency compromises host homeostasis.

LFA-1- and IL-17R-Dependency of the Edil3−/− Periodontitis

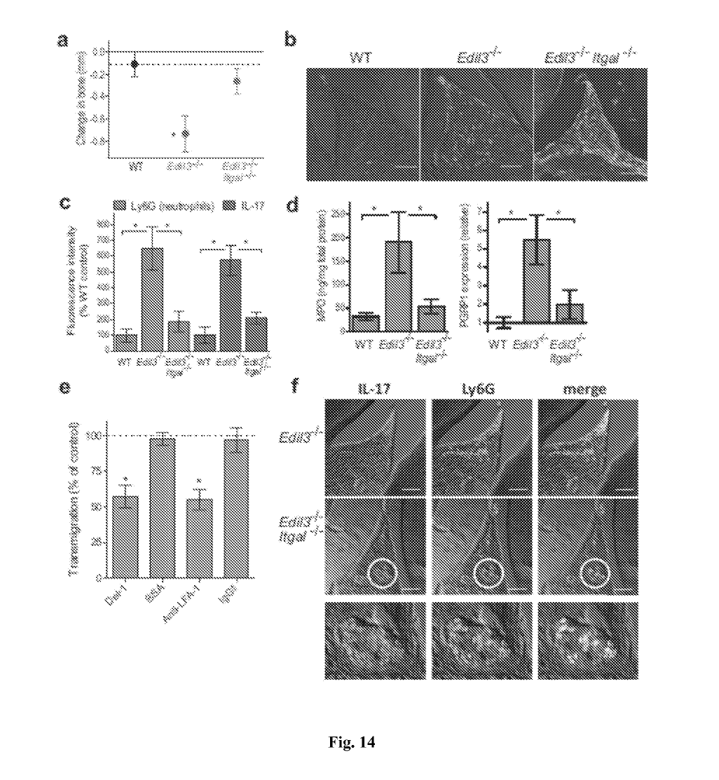

Since Del-1 acts as an antagonist of LFA-1 integrin-dependent neutrophil adhesion (Choi (2008), the present inventors next addressed whether the bone loss seen in Edil3−/− mice could be attributed to increased LFA-1-mediated inflammatory cell recruitment. To this end, the phenotype of 20-week-old mice with combined Del-1 and LFA-1 deficiency (Edil3−/−Itgal−/−) was examined. It was found that the periodontal bone loss associated with Del-1 deficiency was severely inhibited (>75%) in Edil3−/−Itgal−/− mice (FIG. 14a), which additionally had reduced neutrophil infiltration in the gingiva as compared to Edil3−/− mice (FIG. 14b,c). Consistent with the immunohistochemical findings (FIG. 14b), Edil3−/−Itgal−/− mice had reduced amounts of gingival myeloperoxidase (MPO; quantitative marker of neutrophil infiltration) and of the neutrophil-specific peptidoglycan recognition protein-1 (PGRP-1) relative to Edil3−/− mice (FIG. 14d). Mouse Del-1 has been shown to competitively inhibits LFA-1-dependent adhesion of mouse neutrophils to mouse endothelial cells (Choi (2008)). Consistent with the previous findings, human Del-1 also inhibited the LFA-1-dependent transendothelial migration of human neutrophils (FIG. 14e). These data collectively suggest that the protective effect of Del-1 against periodontitis is mediated through regulation of LFA-1-dependent neutrophil trafficking.

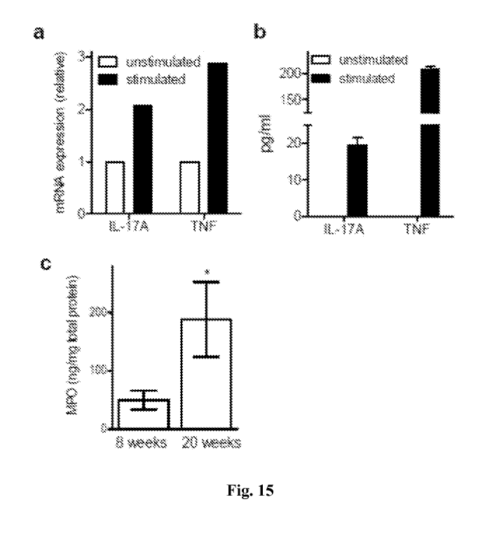

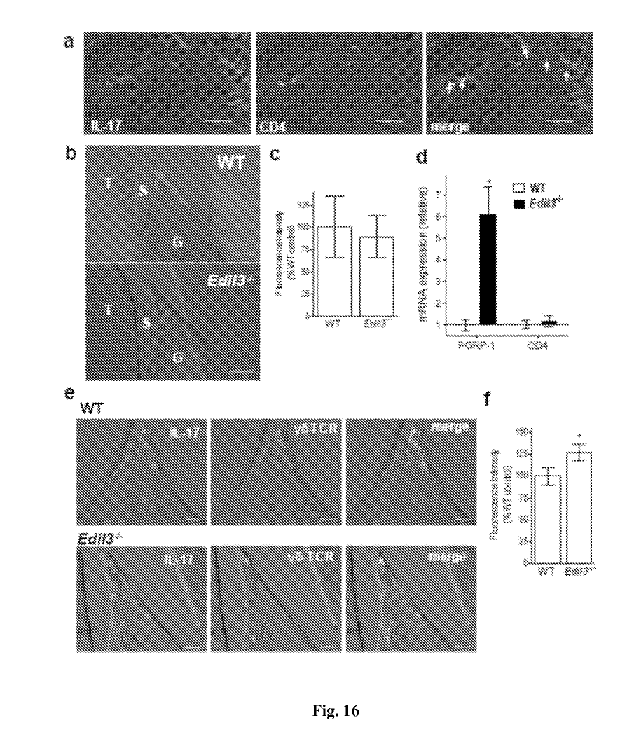

In comparison to Edil3−/− mice, the Edil3−/−Itgal−/− mice displayed decreased gingival elaboration of IL-17 (FIG. 14c,f) due to, at least in part, reduced infiltration of neutrophils which expressed IL-17. In this regard, colocalization of IL-17 and Ly6G (neutrophil marker) in the gingival tissues of both Edil3−/− and Edil3−/−Itgal−/− mice was observed by immunohistochemistry (FIG. 14f). This observation is consistent with the notion that significant portions of the IL-17 released at sites of inflammation derives from innate immune cells, including neutrophils (Cua (2010)). Moreover, it was directly demonstrated that IL-17 mRNA and protein is expressed in bone marrow-isolated mouse neutrophils (FIG. 15a,b), confirming several recent reports (Li (2010), Ferretti (2003), Hoshino (2008), Brodlie (2011), Lin (2011), Eustace (2011)). Although gingival CD4+ T cells also appeared to express IL-17, as indicated by immunohistochemistry (FIG. 16a), their numbers were not elevated in Del-1 deficiency (1 FIG. 16b-d). On the other hand, γδ TCR+ cells, which also produce IL-17 (ref 20), appeared to colocalize with IL-17 in the gingiva and their numbers were modestly but significantly elevated in Edil3−/− mice compared to wild-type mice (FIG. 16e,f). Remarkably, γδ T cells displayed a very high degree of colocalization with IL-17 (80.5%) in the gingiva of 20-week-old Edil3−/− mice, whereas CD4+ T cells had significantly less colocalization with IL-17 (34.2%) (Table 1), consistent with the fact that only a subset of CD4+ T cells is committed to the TH17 lineage. Neutrophils exhibited an intermediate degree of colocalization with IL-17 (65.2%). Strikingly, unlike with the other two cell types, the degree of neutrophil colocalization with IL-17 was significantly elevated with advancing age of Edil3−/− mice (from 17.8% at 8 weeks to 65.2% at 20 weeks) (Table 1). Moreover, the infiltration of neutrophils (as evidenced by elevated MPO amounts) also increased in 20-week-old as compared to 8-week-old Edil3−/− mice (FIG. 15c). These findings suggest that the initial production of IL-17 triggering the recruitment of the first waves of neutrophils may primarily be contributed by other cell types, such as CD4+ T cells and especially γδ T cells, which appear to be an important innate source of IL-17 in the Edil3^{-/-} gingival tissue.

TABLE 1
Quantification of colocalization of various cell types with
IL-17 in the gingival tissue of Edil3^{-/-} mice^{a}.

% Colocalization^{b}

| Cell type (marker used) | 8-wk-old mice | 20-wk-old mice |
|---|---|---|
| γδ T cells (γδ-TCR) | 68.4 ± 12.1^{c} | 80.5 ± 9.7^{d} |
| CD4⁺ T cells (CD4) | 23.3 ± 8.2 | 34.2 ± 10.6 |
| Neutrophils (Ly6G) | 17.8 ± 7.7 | 65.2 ± 14.9^{d,e} |

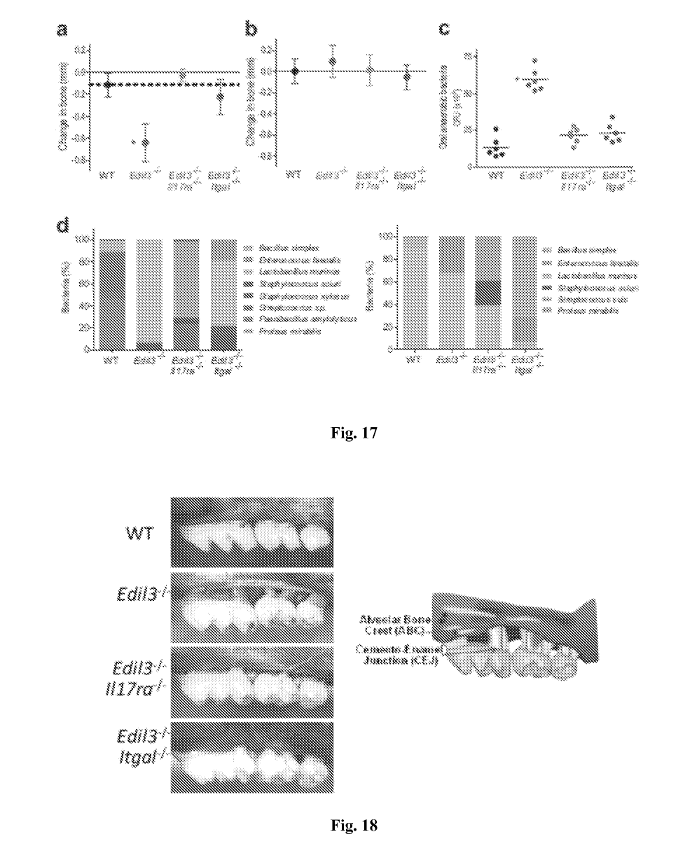

^{a}Colocalization was quantified by means of ImageJ intensity correlation analysis of confocal sections of interdental gingiva, similar to those shown in FIG. 17f and FIG. 16a and 16e.
^{b}Means ± SD (representative confocal images from independent mice; n = 5 mice per group).
^{c}Significantly (p < 0.01) higher compared to CD4⁺ T cells and neutrophils at 8 weeks.
^{d}Significantly (p < 0.01) higher compared to CD4⁺ T cells at 20 weeks.
^{e}Significantly (p < 0.01) higher compared to corresponding 8-wk-old group (no statistically significant differences between the two age groups of CD4+ T cells or γδ T cells).

As Edil3^{-/-} mice expressed abundant IL-17 relative to both wild-type and Edil3^{-/-}Itgal^{-/-} mice, the present inventors then sought to determine the precise role of IL-17 in Del-1 deficiency-associated bone loss. Predicting the role of IL-17 in disease whether protective or destructive is often uncertain since IL-17 can mediate both antimicrobial host defenses and immunopathology (Gaffen (2008), Lubberts (2008)). To conclusively address the role of IL-17, mice with combined Del-1 and IL-17R deficiency were generated. In stark contrast to 20-week-old Edil3^{-/-} mice, age-matched Edil3^{-/-}Il17ra^{-/-} mice were completely protected against bone loss (FIG. 17a and FIG. 18), suggesting that IL-17R signaling is required for induction of periodontal bone loss associated with Del-1 deficiency. Similarly, Il17ra^{-/-} mice had no periodontal phenotype (FIG. 19a); in fact, Il17ra^{-/-} and Edil3^{-/-}Il17ra^{-/-} mice at 30 weeks of age exhibited increased bone heights relative to normal mice (FIG. 19b). These data confirmed that Edil3^{-/-}Itgal^{-/-} mice are protected against periodontitis; in addition, all four genotypes investigated had comparable bone heights in early age (5 weeks) (FIG. 17b) ruling out an innate etiology for the bone loss differences seen amongst the distinct mutant mice at 20 weeks of age (FIG. 17a). Collectively, Del-1 deficiency causes periodontal inflammation and bone loss that is dependent on LFA-1-dependent neutrophil recruitment and IL-17R signaling.

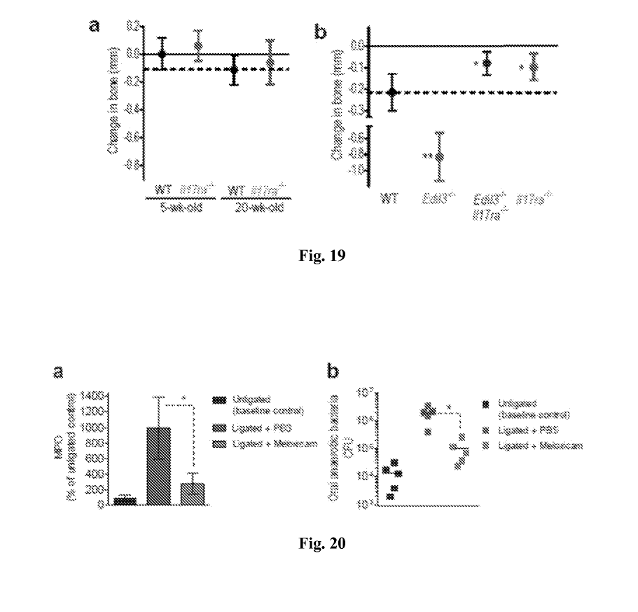

The increased bacterial load due to Del-1 deficiency (FIG. 13a,b) was abrogated in both Edil3^{-/-}Itgal^{-/-} and Edil3^{-/-}Il17ra^{-/-} mice (FIG. 17c). Moreover, relative to their wild-type littermate controls, Edil3^{-/-} mice harbored qualitatively different oral microbiota, the composition of which was further altered in Edil3^{-/-} mice bred with Itgal^{-/-} or Il17ra^{-/-} mice (FIG. 17d), suggesting that host genetics may determine the composition of the host-associated microbiota. The fact that each mouse genotype, whether resistant or susceptible to periodontitis, harbored qualitatively different oral microbiota suggests that compositional shifts away from the wild-type microbiota are not necessarily involved in disease pathogenesis. However, the observed changes to the microbiota in the presence of overt inflammation (as seen in Del-1 deficiency) are consistent with findings that certain oral biofilm species thrive under excessive inflammation, which generates tissue breakdown products that serve their nutritional needs³¹. This notion is further supported by observations that anti-inflammatory treatment of mice with meloxicam (selective cyclooxygenase-2 inhibitor) could reduce the bacterial load even though it also reduced neutrophil infiltration (FIG. 20). Therefore, destructive inflammation may actually support the overgrowth of periodontal bacteria despite recruitment of high numbers of neutrophils, consistent with the findings that the high bacterial burden associated with Del-1 deficiency was restored to near normal numbers in Edil3^{-/-}Itgal^{-/-} and Edil3^{-/-}Il17ra^{-/-} mice.

IL-17 Regulates Del-1 Expression

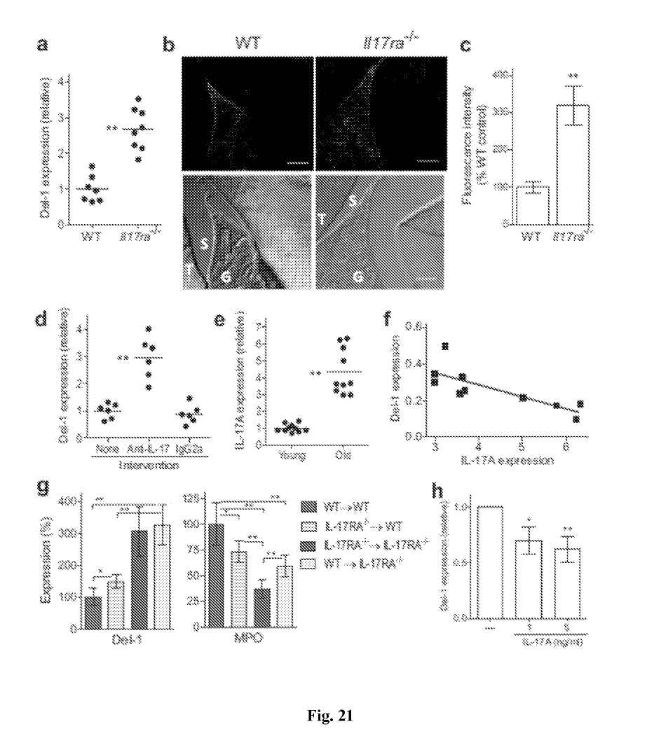

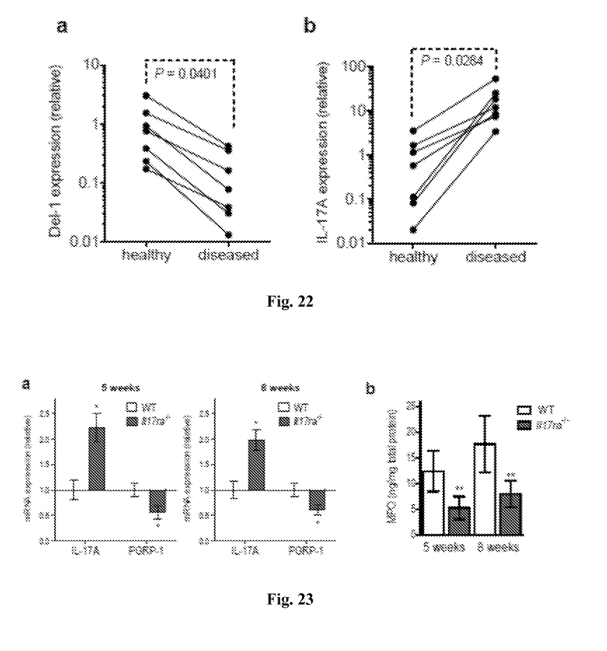

As IL-17 can orchestrate the production, recruitment, and activation of neutrophils during inflammation (Kolls (2004), Stark (2005)), the present inventors next examined whether IL-17 can additionally regulate Del-1 expression. It was found that Il17ra^{-/-} mice exhibited increased gingival Del-1 mRNA and protein expression relative to wild-type controls (FIG. 21a-c). Moreover, local microinjection of anti-IL-17 mAb into the gingiva of old mice resulted in significant upregulation of Del-1 expression, whereas an isotype control had no effect (FIG. 21d). Furthermore, the gingival expression of IL-17 was enhanced in old age (FIG. 21e), in stark contrast to the decreased Del-1 expression in old mice (FIG. 9b). Linear-regression analysis of Del-1 expression versus IL-17 expression (data from FIGS. 9b and 21e, respectively, involving same set of 18-month-old mice) revealed a significant inverse association between IL-17 and Del-1 (r²=0.6274, P=0.0063; FIG. 21f). Consistent with these findings, diseased (inflamed) gingival sites from human periodontitis patients expressed significantly more IL-17A and correspondingly less Del-1 mRNA as compared to control healthy sites from the same individuals (FIG. 22). Therefore, the inverse association between Del-1 and IL-17A expression characterizes also the human periodontium.

To determine the contribution of local IL-17R signaling in Del-1 regulation, the following combinations of bone marrow (BM) chimeric mice (donor BM→lethally irradiated recipient) were generated: WT→WT, Il17ra^{-/-}→WT, Il17ra^{-/-}→Il17ra^{-/-}, and WT→Il17ra^{-/-}. Six weeks after BM reconstitution, Il17ra^{-/-} recipient mice had significantly higher gingival Del-1 expression than WT recipient mice, regardless of whether they received WT or Il17ra^{-/-} BM (FIG. 21g, left). Therefore, high Del-1 expression correlates with lack of IL-17R signaling on stromal cells. Interestingly, Il17ra^{-/-}→WT mice displayed a modest increase in Del-1 expression compared to WT→WT (FIG. 21g, left).

Strikingly, MPO amounts were highest in WT→WT mice and were incrementally decreased in Il17ra^{-/-}→WT and Il17ra^{-/-}→Il17ra^{-/-} mice following the inverse pattern of Del-1 expression (FIG. 21g). IL-17R signaling on hematopoietic cells contributes to the regulation of neutrophil recruitment (though not as potently as IL-17R signaling on stromal cells) (Smith (2008)), possibly because IL-17 can directly stimulate the chemotactic recruitment of neutrophils (Lemos (2009)). Consistently, neutrophil recruitment, assessed by measuring gingival MPO amounts, was higher in WT→Il17ra^{-/-} mice as compared to Il17ra^{-/-}→Il17ra^{-/-} mice, whereas Il17ra^{-/-}→WT mice had lower MPO amounts as compared to WT→WT mice (FIG. 21g, right). Reduced neutrophil infiltration could thus cause a reduction in local IL-17 production accounting for the increased Del-1 expression (FIG. 21g, left).

Therefore, gingival Del-1 expression is regulated by IL-17R signaling predominantly on stromal cells and is strongly correlated with neutrophil recruitment to the gingival tissue. The stromal cells involved are most likely endothelial cells since gingival Del-1 expression could be localized specifically to endothelial cells (FIG. 8). Consistent with the ability of IL-17 to inhibit endothelial expression of Del-1 in mouse gingiva, human IL-17A inhibited Del-1 expression in human endothelial cells (FIG. 21h).

Although Il17ra^{-/-} mice displayed reduced neutrophil recruitment to the gingiva at very young age, they expressed high amounts of IL-17A compared to age-matched wild-type controls (FIG. 23), consistent with a previous independent study (Smith (2008)). This early expression of IL-17 in the gingiva of Il17ra^{-/-} mice, in the absence of significant neutrophil infiltration, further supports (see also FIG. 15c and Table 1) the notion that the gingival tissue contains IL-17-expressing cells, other than neutrophils, which may form a source of early IL-17 production for the recruitment of the first waves of neutrophils.

Administration of Del-1 Inhibits Inflammatory Bone Loss

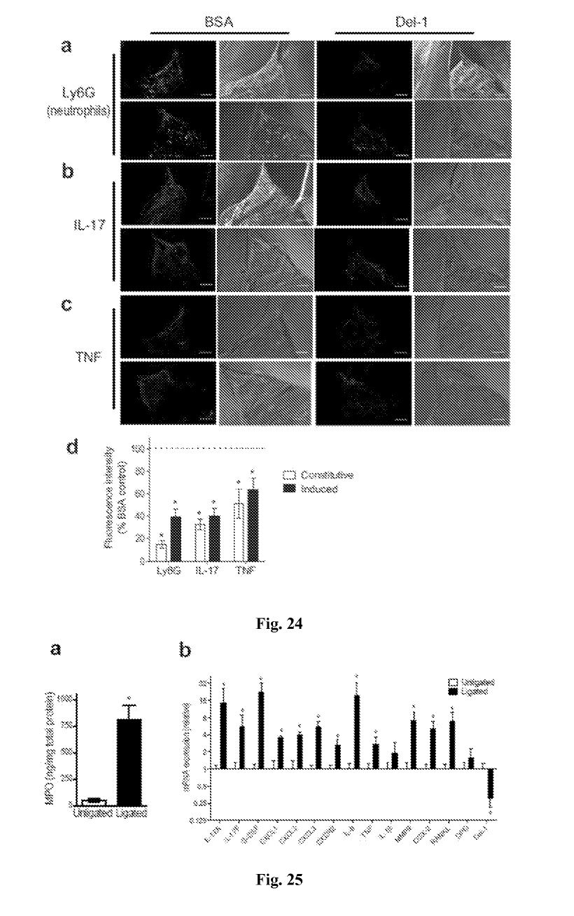

It was next determined whether recombinant soluble Del-1 could be exploited therapeutically to reverse periodontal inflammation in old mice, which are practically deficient in Del-1. Indeed, local gingival microinjection of Del-1 resulted in reduced neutrophil infiltration and diminished expression of IL-17 and TNF (FIG. 24a-c) in the periodontium compared to similar treatment with BSA control (FIG. 24d). Notably, Del-1 treatment of old mice suppressed both constitutive (naturally occurring) inflammation (FIG. 24a-c; top) as well as inflammation induced by exogenous oral inoculation with the human pathogen Porphyromonas gingivalis (FIG. 24a-c; bottom). The ability of Del-1 to reduce expression of IL-17 and TNF protein was confirmed at the mRNA level by qPCR, which additionally revealed reduced transcript abundance of other proinflammatory cytokines, chemokines, chemokine receptors, pattern-recognition and complement receptors, and costimulatory molecules (Table 2).

TABLE 2
Reduced mRNA expression of inflammatory mediators in the gingiva
of aged mice after local administration of soluble Del-1 ^{a}.

Fold change vs. Del-1-untreated controls

| Molecule | CMC | Pg |
|---|---|---|
| IL-17 | 0.19 | 0.25 |
| TNF | 0.50 | 0.51 |
| IL-6 | 0.25 | 0.24 |
| IL-1 | 0.50 | 0.25 |
| RANKL | 0.49 | 0.25 |
| G-CSF | 0.12 | 0.25 |
| CCL2 | 0.48 | 0.49 |
| CCL3 | 1.02 | 0.51 |
| CCL20 | 0.24 | 0.49 |
| CXCL10 | 0.25 | 0.26 |
| CCR2 | 0.26 | 0.12 |
| CCR6 | 0.25 | 0.13 |
| CXCR4 | 0.50 | 0.24 |
| C3aR | 0.49 | 0.49 |
| C5aR | 0.50 | 0.25 |
| TREM-1 | 0.49 | 0.24 |
| TLR1 | 0.24 | 0.24 |
| TLR2 | 0.26 | 0.25 |
| TLR4 | 0.25 | 0.12 |
| TLR5 | 0.51 | 0.51 |
| TLR6 | 0.27 | 0.26 |
| TLR9 | 0.25 | 0.51 |
| CD40 | 0.24 | 0.25 |
| CD80 | 0.25 | 0.25 |
| CD86 | 0.50 | 0.49 |

^{a} Eighteen-month-old C57BL/6 mice were microinjected with BSA (control) or Del-1 in the palatal gingiva between the first and the second molar teeth. The mice were then orally inoculated with 10⁹ CFU Porphyromonas gingivalis in 2% carboxymethylcellulose (CMC) or CMC only, and were sacrificed 12 h later. The interdental gingiva (i.e., between first and second molar teeth) were dissected and processed for qPCR to determine gingival mRNA expression of the indicated molecules (normalized against GAPDH mRNA and expressed as fold change of Del-1-treated relative to corresponding BSA-treated controls). To ensure enough tissue material, dissected interdental gingiva from 5 mice per group were pooled before use in the qPCR. Consistent results were obtained from an identical independent experiment.

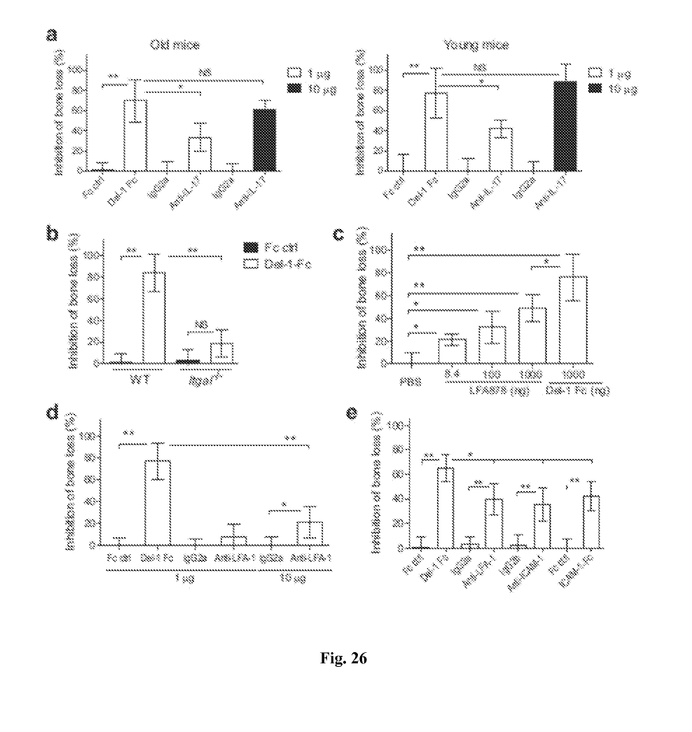

It was then investigated whether Del-1 could inhibit bone loss. Because naturally induced bone loss is a slow process and long-term delivery of Del-1 in mice until old age would not be practically feasible, the ligature-induced periodontitis model' was used. In this model, a silk ligature is placed around molar teeth resulting in massive local bacterial accumulation and induction of rapid bone loss in conventional (but not germ-free) rodents (Graves (2008)). It was confirmed that this model leads to neutrophil recruitment to the periodontium (as revealed by pronounced elevation of MPO amounts), accompanied by upregulation of inflammatory markers such as IL-17A, chemokines, and RANKL, and by downregulation of Del-1 (FIG. 25). Therefore, this is an appropriate model to test whether treatment with Del-1 can inhibit inflammatory bone loss. To this end, Del-1, expressed as a fusion protein with human IgG Fc (which may increase Del-1 bioavailability in the tissue), was microinjected in the gingiva one day before placement of the ligature and every day thereafter until the day before sacrifice on day 5. In contrast to Fc control, which had no significant effects, Del-1-Fc inhibited the induction of bone loss by in 18-month-old mice and by ≈80% in 10-week-old mice relative to no-treatment (FIG. 26a). The reversal of periodontitis in Edil3^{-/-}Il17ra^{-/-} mice relative to Edil3^{-/-} mice suggested that IL-17 could mediate induction of bone loss. Indeed, local administration of mAb to IL-17A inhibited ligature-induced bone loss in both young and old mice (FIG. 26a).

In stark contrast to its potent protective effect in normal LFA-1-sufficient mice, Del-1-Fc treatment had a minor, but not statistically significant, effect against bone loss in Itgal^{-/-} mice (FIG. 26b). This finding is consistent with the significant inhibition of periodontitis in Edil3^{-/-}Itgal^{-/-} mice, relative to Edil3^{-/-} mice, and establishes that the protective effect of Del-1 requires the presence of LFA-1 on the inflammatory cells.

Del-1-Fc was next compared with other treatments that can block LFA-1 interactions. Locally administered LFA878, a potent small-molecule LFA-1 inhibitor (Weitz-Schmidt (2004)), conferred protection almost (but not quite) comparable to Del-1-Fc when given at 1 μg; however, LFA878 was less protective than Del-1-Fc when the two molecules were compared at equal molar amounts (8.4 ng and 1 μg, respectively) (FIG. 26c). Local administration of the M17/4 anti-LFA-1 mAb (Choi (2008)) was modestly protective against bone loss when given at a 10-fold higher dose than Del-1-Fc (FIG. 26c), although its efficacy approached that of Del-1-Fc when both inhibitors were given systemically (FIG. 26d). Systemic treatments with anti-ICAM-1 mAb or ICAM-1-Fc conferred protection against bone loss comparable to that seen with anti-LFA-1, but significantly less effective than Del-1-Fc (FIG. 26e). In these experiments, treatments with Fc or isotype controls consistently had no effect on bone loss (FIG. 26d-f). In summary, Del-1-Fc, given locally or systemically, appears to be more potent in inhibiting periodontal bone loss than other inhibitors that interfere with the LFA-1-ICAM-1 interaction.

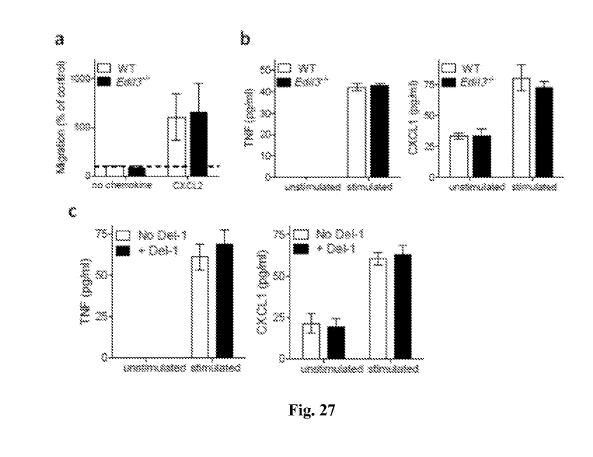

The LFA-1 dependence of the protective effect of Del-1 (FIG. 26b) is consistent with the notion that Del-1 acts by regulating neutrophil recruitment, whereas its absence (Edil3^{-/-} mice) leads to periodontitis (FIG. 14a). However, these data cannot formally rule out that Del-1 deficiency may also have direct effects on neutrophils. Arguing against this notion are the observations that neutrophils isolated from wild-type or Edil3^{-/-} mice had comparable intrinsic capacity for migration and cytokine or chemokine induction (FIG. 27a,b). Moreover, Del-1 did not exert a direct effect on cytokine and chemokine induction by neutrophils (FIG. 27c). Collectively, these findings provide proof-of-concept that Del-1 has therapeutic potential for the treatment of periodontal inflammation and bone loss, and perhaps other neutrophil-mediated inflammatory diseases.

Discussion

As disclosed herein, Del-1 serves a mechanism by which a tissue self-regulates the local inflammatory response to prevent immunopathology. Specifically, Del-1 is required for homeostatic inhibition of inflammatory periodontal bone loss, which involves LFA-1-dependent neutrophil recruitment and IL-17R signaling. Importantly, Edil3^{-/-} mice developed periodontitis naturally in a chronic setting of dysregulated neutrophil recruitment, without any experimental intervention as often required in animal periodontitis models (e.g., infection with a human pathogen or injection of bone loss-inducing agents) (Graves (2008)).

The Edil3^{-/-} phenotype was mirrored in normal old mice, where inflammatory bone loss was correlated with diminished Del-1expression and elevated IL-17 expression. In this regard, gingival Del-1 expression was downregulated by IL-17R signaling acting on endothelial cells. Previously, it was shown that IL-17 promotes granulopoiesis and induces the chemotactic recruitment, activation, and survival of neutrophils (Gaffen (2008), Kolls (2004), Stark (2005)). Now a novel mechanism is shown, by which IL-17 facilitates neutrophil recruitment and promotes inflammation, namely through downregulation of the endogenous anti-inflammatory factor Del-1. This function may be beneficial in the acute defense against infection, although persistent recruitment and infiltration of neutrophils into peripheral tissues may contribute to the pathogenesis of periodontitis⁷⁻⁸ and other chronic inflammatory diseases (Luster (2005), Cascao (2010), Hartl (2008)).

The selective recruitment of neutrophils in Del-1 deficiency can be attributed, in large part, to the highly elevated expression of IL-17, which predominantly recruits neutrophils (Witowski (2000). Moreover, the restricted expression pattern of Del-1 likely confers its tissue-specific anti-inflammatory activity (Chavakis (2009)). This role of Del-1 is in line with recent findings that growth differentiation factor-15, locally produced in the heart, protects the infarcted myocardium from excessive neutrophil infiltration by inhibiting integrin activation (Kempf (2011)). This recent study and the current findings support an emerging concept that tissues have evolved distinct local homeostatic mechanisms to control inflammatory cell recruitment and prevent tissue damage.

These findings also represent the first causal link between IL-17 and periodontal bone loss, consistent with the elevated expression of IL-17 in human periodontitis (Gaffen (2008), Ohyama (2009)). IL-17R signaling also stimulates antimicrobial immunity (Dubin (2008) and was associated with protection in a model of periodontitis induced by implantation of a human pathogen (Yu (2007)). In a pathological context, however, IL-17 can mediate connective tissue destruction and bone resorption via induction of matrix metalloproteases and RANKL (Lubberts (2008)). Consistently, the higher expression of IL-17 in Del-1-deficient mice was accompanied by increased periodontal RANKL production and osteoclastic activity.

The recent demonstration that neutrophils express RANKL underscores their potential to directly engage in inflammatory bone destruction (Chakravarti (2009)). In line with this, much of the IL-17 in inflammatory sites is actually contributed by neutrophils, as also shown recently by others (Li (2010), Ferretti (2003), Hoshino (2008), Brodlie (2011), Lin (2011), Eustace (2011)), and other innate immune cells such as γδ T cells, although IL-17 is a signature cytokine of the CD4⁺ T-helper 17 subset (Cua (2010)). Although colocalization of IL-17 with both neutrophils and CD4⁺ cells in Edil3^{-/-} gingiva was observed, the numbers of CD4⁺ cells were not elevated as observed with neutrophils. Innate γδ T cells (Cua (2010)) also colocalized with IL-17 and their numbers were significantly but modestly elevated in Edil3^{-/-} gingiva. Strikingly, γδ T cells exhibited very high colocalization with IL-17, as compared to CD4⁺ T cells or neutrophils, although the latter approached the degree of γδ T cell colocalization with IL-17 several weeks after the onset of the disease (at 20 weeks, when bone loss becomes pronounced). By contrast, in the early stages (8 weeks) of Del-1 deficiency-associated periodontitis, there was relatively low infiltration of neutrophils which only modestly colocalized with IL-17. These findings suggest that the initial source of IL-17 for the recruitment of the first waves of neutrophils may primarily be contributed by other cell types, such as CD4⁺ T cells or, more likely, γδ T cells, consistent with their proposed function as first-line defense and immunoregulatory cells in the human gingiva (Lundqvist (1994). Therefore, while neutrophils may represent the main effector cells contributing to inflammatory bone loss, other gingival cells, particularly γδ T cells, may represent the initial trigger in a manner dependent on IL-17. By virtue of their high numbers and ability to express IL-17 at later stages of the disease, neutrophils may eventually become an important source of IL-17 that contributes to the perpetuation of neutrophil recruitment and the inflammatory periodontal bone destruction.

The term ‘inflamm-aging’ was aptly coined to describe the heightened chronic inflammatory state often associated with old age in humans (Cevenini (2010)). In this regard, the elderly show inappropriately high periodontal inflammatory responses relative to young individuals following comparable de novo periodontal biofilm formation (Hajishengallis (2010)). From a mechanistic viewpoint, little is known regarding the impact of aging on innate immunity and inflammatory diseases (Hajishengallis (2010), Gomez (2008)). However, the reduced expression of Del-1 could be a major mechanism linking advanced age to destructive periodontal inflammation.

The majority of adults experience some form of periodontal disease and an estimated 10-15% develops severe periodontitis, which is a risk factor for systemic conditions (Pihlstrom (2005), Genco (2010), Tonetti (2007), Lundberg (2010), Lalla (2011)). Conventional periodontal treatment is often not sufficient to control destructive inflammation and many patients develop recurrent disease (Armitage (2002)). The findings support the feasibility of controlling the influx of neutrophils and ensuing IL-17-dependent inflammation through Del-1 treatment. As an endogenous anti-inflammatory factor, Del-1 may be a safe and promising approach to treat periodontitis and reduce the risk for associated systemic diseases and, moreover, may find application in other inflammatory and autoimmune diseases.

Methods

Mice.

All animal procedures were approved by the University of Louisville Institutional Animal Care and Use Committee, in compliance with established federal and state policies. C57BL/6 Il17ra^{-/-} and Itgal^{-/-} mice were generously provided, respectively, by Amgen (Seattle, Wash.) and C. M. Ballantyne (Baylor College of Medicine). The generation of C57BL/6 Edil3^{-/-} and Edil3^{-/-}Itgal^{-/-} mice was previously described (Choi (2008)). In this study, Edil3^{-/-} and Il17ra^{-/-} mice were crossed to generate double knock-outs (Edil3^{-/-}Il17ra^{-/-}). In aging experiments, knockout mice and wild-type littermate controls were reared in parallel under specific-pathogen-free conditions Chimeric mice were generated by adoptive transfer of donor bone marrow (BM) cells into lethally irradiated recipient mice (950 rads of total-body irradiation). The BM cells, harvested by flushing both femurs and tibias of donor mice, were injected at 5×10⁶ into each recipient mouse. The following combinations (donor BM→lethally irradiated recipient) were generated: WT→WT, Il17ra^{-/-}→WT, Il17ra^{-/-}→Il17ra^{-/-}, and WT→Il17ra^{-/-}. The mice were used for experiments 6 weeks after BM reconstitution.

Determination of Periodontal Bone Loss.

Periodontal bone heights were assessed in defleshed maxillae under a dissecting microscope (×40) fitted with a video image marker measurement system (VIA-170K; Boeckeler Instruments). The CEJ-ABC distance was measured on 14 predetermined maxillary sites (Baker (2000)). To calculate relative bone loss (e.g., Edil3^{-/-} mice vs. wild-type controls, or old mice vs. young controls), the 14-site total CEJ-ABC distance for each mouse was subtracted from the mean CEJ-ABC distance of control mice. The results were expressed in mm and negative values indicated bone loss relative to controls (Baker (2000)).

The ligature-induced periodontitis model was used to determine the efficacy of potential therapeutic interventions (see below). Specifically, bone loss was induced by tying a 5-0 silk ligature around the maxillary left second molar, placing the ligature in the gingival sulcus; this treatment induces bone loss in conventional (but not germ-free) mice due to massive bacterial accumulation in the ligated teeth (Graves (2008)). The contralateral molar tooth in each mouse was left unligated (baseline control). Bone loss was examined 5 days after placement of the ligatures, which remained in place in all mice during the experimental period. Using the VIA-170K system, bone measurements were performed on the ligated second molar (3 sites corresponding to mesial cusp, palatal groove, and distal cusp) and the affected adjacent regions (sites corresponding to distal cusp and distal groove of the first molar, and palatal cusp of the third molar). To calculate bone loss, the 6-site total CEJ-ABC distance for the ligated side of each mouse was subtracted from the 6-site total CEJ-ABC distance of the contralateral unligated side of the same mouse.

Intervention Experiments.

Ten 18-month-old C57BL/6 mice were microinjected in the gingiva with soluble recombinant mouse Del-1 (kindly provided by Valentis, Inc.)⁴ and another ten mice with BSA control. Specifically, Del-1 or BSA (1 μg in 1-μl volume) were microinjected through a 28.5-gauge MicroFine needle (BD) into the palatal gingiva between the first and the second molar teeth, on both sides of the maxilla. Half of the Del-1 or BSAtreated mice were additionally orally inoculated with P. gingivalis ATCC 33277 (10⁹ CFU) in 2% carboxymethylcellulose vehicle and the other half were orally given vehicle alone. All mice were sacrificed 12 h later. Maxillae were harvested and one side was stored in 4% paraformaldehyde for immunohistochemistry (see below), while the other side was used to dissect interdental gingiva which were placed into RNAlater solution (Ambion) for qPCR (see below). To determine whether IL-17 regulates Del-1 expression, a neutralizing anti-IL-17A mAb (clone M210, rat IgG2a; kindly provided by Amgen) was microinjected into the palatal gingiva (1 μg) as described above. Purified azide-free rat IgG2a (Biolegend) served as control. qPCR was used to determine IL-17 mRNA expression in dissected gingiva.

To determine its protective efficacy in ligature-induced periodontitis, Del-1 was used in the form of a fusion protein with the Fc region of human IgG1 (Del-1-Fc) (GenScript). Del-1-Fc (1 μg) was microinjected into the palatal gingiva of the ligated second maxillary molar, one day before placement of the ligature and every day thereafter until the day before sacrifice on day 5. In other experiments, Del-1-Fc was administered systemically by i.v. injection (50 μg), following the same timing schedule as above. The same protocols were used to determine the effect of local (or systemic) administration of mAbs to LFA-1 (clone M17/4, rat IgG2a; BioLegend), ICAM-1 (clone YN1, IgG2b; Biolegend), IL-17A (clone M210, rat IgG2a; Amgen), or IL-17F (clone 316016, rat IgG2a; R&D Systems). Recombinant mouse ICAM-1 fused to the Fc region of human IgG1 (ICAM-1-Fc) as well as an LFA-1 antagonist (LFA878; kindly provided by Novartis) (Weitz-Schmidt (2004)) were also used in bone loss inhibition experiments. Purified azide-free rat IgG2a or IgG2b (BioLegend) and recombinant human IgG1 Fc (R&D Systems) were used as controls for the mAbs and Del-1-Fc, respectively.

Oral Bacterial Sampling and Identification.

The murine oral cavity was sampled for 30 s using sterile fine tip cotton swabs held against the gum lines (Baker (2000)). Serial dilutions of the swab extracts were plated onto blood agar plates for aerobic and anaerobic growth and CFU determination. In certain experiments, the cultivatable bacteria were purified by subculture and identified by MALDI Biotyper (Bruker Daltonics) and by 16S ribosomal RNA sequencing in some cases (Hajishengallis (2011)).

Statistical Analysis.

Data were evaluated by analysis of variance and the Dunnett multiple-comparison test using the InStat program (GraphPad Software, San Diego, Calif.). Where appropriate (comparison of two groups only), two-tailed t tests were performed. P<0.05 was considered to be significant. All experiments were performed at least twice for verification.

Additional Methods

Immunohistochemistry.

Maxillae with intact surrounding tissue were fixed in 4% paraformaldehyde, decalcified in Immunocal solution (Decal) for 15 days, and embedded in OCT compound. Serial mesio-distal sections (7- to 8-μm thick) parallel to the long axis of the teeth (sagittal) were stained with monoclonal antibodies to mouse Ly6G (RB6-8C5, FITC-conjugate; LifeSpan BioSciences), mouse CD4 (GK1.5, FITC- or PE-conjugate; LifeSpan), mouse γδ T-cell receptor (GL3, FITC-conjugate; LifeSpan Biosciences) or with polyclonal antibodies to human/mouse Del-1 (ProteinTech), human/mouse IL-17A (Santa Cruz Biotech), human/mouse TNF (Abcam), human/mouse RANKL (Santa Cruz), and mouse CD31 (LifeSpan). Where necessary, staining involved the use of a secondary reagent (AlexaFluor594-conjugated goat anti-rabbit IgG; Molecular Probes). The specificity of staining was confirmed by using appropriate FITC-conjugated isotype controls or normal rabbit IgG followed by AlexaFluor594-goat anti-rabbit IgG. Images were captured using a laser-scanning confocal microscope (Olympus FV1000). Fluorescence intensity was quantified using the Image J software (NIH; http://rsb.info.nih.gov/ij).

Colocalization Analysis.

The degree of colocalization of various cell types with IL-17 in confocal sections of interdental gingiva was quantified using the public domain Image J software with the Intensity Correlation Analysis plugin (NIH; http://rsb.info.nih.gov/ij).

Histological TRAP Staining.

Upper jaws (maxillae) with intact surrounding tissue were fixed in 4% paraformaldehyde, decalcified in Immunocal solution (Decal Chemical Corp.) for 15 days, and embedded in OCT compound. Osteoclasts were identified in mesio-distal sections (7- to 8-μm thick) of maxillae, using tartrate-resistant acid phosphatase (TRAP) staining This was carried out using the leukocyte acid phosphatase kit as per the manufacturer's protocol (Sigma Aldrich). Slides were viewed using a Nikon E800 microscope. TRAP⁺ multinucleated cells lying along the alveolar bone surface were considered to be osteoclasts.

Histochemical Detection of β-Galactosidase Enzymatic Activity.

β-galactosidase activity in the gingiva of Edil3^{-/-} mice (Del-1 knock-out/LacZ knock-in transgenics) was detected using a β-galactosidase staining kit (Minis). Gingival specimens, collected from mouse maxillae, were frozen at −80° C. in OCT compound until sectioning. For staining, 7- to 8-μm thick frozen gingival tissue sections were conditioned in PBS, fixed with 2% glutaraldehyde, and stained with X-gal solution, using the manufacturer's protocol. Images were acquired using a Nikon E800 microscope. β-galactosidase was alternatively detected by confocal immunohistochemistry (see Immunohistochemistry in Example) using a mAb to β-galactosidase (Clone GAL-13) from Sigma-Aldrich.

MPO Assay.

The concentration of MPO in gingival tissue homogenates was determined using an ELISA kit according to the manufacturer's instructions (Hycult Biotechnology). MPO concentrations were normalized to the total protein concentrations in the tissue homogenates, as measured using the Coomassie Plus Bradford protein assay kit (Pierce).

Quantitative Real-Time PCR (qPCR).

Total RNA was extracted from excised gingival tissue or cultured cells using the PerfectPure RNA cell kit (5 Prime, Fisher) and quantified by spectrometry at 260 and 280 nm. The RNA was reverse-transcribed using the High-Capacity cDNA Archive kit (Applied Biosystems) and qPCR with cDNA was performed using the ABI 7500 Fast System, according to the manufacturer's protocol (Applied Biosystems). TaqMan probes, sense primers, and antisense primers for qPCR of genes investigated in this paper were purchased from Applied Biosystems.

Isolation of Mouse Neutrophils.

Mouse neutrophils were obtained by harvesting the bone marrow (Ley (2007)). Briefly, bone marrow cells, obtained after flushing tibias and femurs with RPMI 1640 containing 10% FBS, were depleted of tissue debris and erythrocytes and were resuspended in 45% Percoll. The cell suspension was then overlaid onto a four-layer Percoll gradient (50%, 55%, 62% and 81%) and centrifuged at 1,200 g for 30 min at 4° C. Mature neutrophils were collected at the 81% interface and washed twice in PBS. Neutrophil purity was routinely >95%, as determined by FACS after staining for Ly6G, F4/80, and CD3.

Bone Loss in Antibiotic-Treated Mice.

In certain experiments, bone loss (determined as outlined in herein under Determination of periodontal bone loss) was measured in mice which were administered antibiotics in their drinking water. Specifically, the mice were provided water supplemented with sulfamethoxazole and trimethoprim at a final concentration of 800 μg/ml and 400 μg/ml, respectively. Control mice were given plain water.

Transmigration Assay.

Transmigration assays were performed as previously described (Ley (2007)), using 6.5-mm Transwells with an 8-μm pore size (Corning). Briefly, human umbilical vein endothelial cells (HUVEC; PromoCell) were seesed on Transwell filters 2 days prior to the assay and grown without medium in the lower compartment for 48 h in a humidified atmosphere (37° C., 5% CO₂). Neutrophils isolated from human peripheral blood were added to the upper well and were allowed to migrate towards migration-assay medium (serum-free RPMI with 0.3% BSA in the absence or presence of 20 ng/ml CXCL8) in the lower compartment of the Transwell system. After 60 min incubation at 37° C., the number of transmigrated cells in the lower compartment was measured (Ley (2007)). Soluble human Del-1 used in these experiments was from R&D Systems. Human blood collections were conducted in compliance with established guidelines approved by the Institutional Review Board of the University of Louisville.

Chemotaxis.

The chemotaxis of primary neutrophils isolated from the bone marrow of wild-type or Edil3^{-/-} mice towards 10 ng/ml of CXCL2 was tested using a Transwell system with 5-μm pores (Corning) according to a previously described protocol (Luster (2005)).

Peripheral Quantitative Computer Tomography (pQCT).

The left femur and lumbar spine, stored in 70% ethanol, were used for bone mineral density (BMD) measurements. BMD of the distal femoral metaphysis and diaphysis was measured by pQCT using a XCT Research M+pQCT machine (Stratec Medizintechnik). The measurements were made with a voxel size of 70 μm. One slice in the mid-diaphysis of the femur located 7 mm distal from the growth plate, and three slices in the femoral metaphysis located 1 mm distal from the growth plate were measured. The position of the growth plate was determined with the help of scout view image. For the measurement of trabecular BMD, regions of interest were set and contour mode 1 and peel mode 2 were used. The threshold for the trabecular bone was 230 mg/cm³ and the threshold for cortical BMD was 710 mg/cm³.

Human Gingival Tissue Samples:

Gingival tissue samples were obtained during periodontal surgery under institutional review board approval. The disease status was determined according to the Armitage periodontal disease classification (Chavakis (2009)). The patients (4 males and 3 females, age range between 40 and 50) presented localized severe chronic periodontitis. Patients with uncontrolled diabetes, previous head and neck radiation therapy, chemotherapy in the previous 12 months immune diseases or other systemic diseases that significantly affect the periodontium, were excluded from the study. Smoker and pregnant patients were also excluded from the study. Diseased (inflamed) tissues were collected around disease involved-teeth and corresponded to attachment loss≧5 mm and probing depth≧5 mm. Control sites were free of apparent periodontal inflammation and they corresponded to zero attachment loss and probing depth≦3 mm; these were diagnosed as periodontally healthy sites and were separated by at least one tooth from periodontal inflammatory lesions.

### SEQUENCES

In studies related to the findings disclosed herein, a Del-1 Polypeptide was expressed as a fusion protein with IgG1 Fc. With reference to the amino acid sequence of SEQ ID NO: 5, the Del-1 polypeptide—amino acid residues 1-480; IgG1 Fc—amino acid residues 495-721 (see also SEQ ID NO: 3, encoded by the nucleic acid of SEQ ID NO: 2); Myc Epitope—amino acid residues 727-741; Histag—amino acid residues 742-747; residues supplied by vector (pSecTag2, Invitrogen)—amino acid residues 481-494 and 722-726. The cDNA encoding the polypeptide of SEQ ID NO: 5 is set forth in SEQ ID NO: 6.

Human Del-1 Amino Acid
SEQ ID NO: 1
MKRSVAVWLLVGLSLSVPQFGKGDICDPNPCENGGICLPGLADGSFSC
ECPDGFTDPNCSSVVEVASDEEEPTSAGPCTPNPCHNGGTCEISEAYR
GDTFIGYVCKCPRGFNGIHCQHNINECEVEPCKNGGICTDLVANYSCE
CPGEFMGRNCQYKCSGPLGIEGGIISNQQITASSTHRALFGLQKWYPY
YARLNKKGLINAWTAAENDRWPWIQINLQRKMRVTGVITQGAKRIGSP
EYIKSYKIAYSNDGKTWAMYKVKGTNEDMVERGNIDNNTPYANSFTPP
IKAQYVRLYPQVCRRHCTLRMELLGCELSGCSEPLGMKSGHIQDYQIT
ASSIFRTLNMDMFTWEPRKARLDKQGKVNAWTSGHNDQSQWLQVDLLV
PTKVTGIITQGAKDFGHVQFVGSYKLAYSNDGEHWTVYQDEKQRKDKV
FQGNFDNDTHRKNVIDPPIYARHIRILPWSWYGRITLRSELLGCTEEE
Human Del-1 Nucleic Acid Sequence
SEQ ID NO: 2
atgaagcgctc ggtagccgtc tggctcttgg tcgggctcag
cctcagtgtc ccccagttcg gcaaaggtga tatttgtgat
cccaatccat gtgaaaatgg aggtatctgt ttgccaggat
tggctgatgg ttccttttcc tgtgagtgtc cagatggctt
cacagacccc aactgttcta gtgttgtgga ggttgcatca
gatgaagaag aaccaacttc agcaggtccc tgcactccta
atccatgcca taatggagga acctgtgaaa taagtgaagc
ataccgaggg gatacattca taggctatgt ttgtaaatgt
ccccgaggat ttaatgggat tcactgtcag cacaacataa
atgaatgcga agttgagcct tgcaaaaatg gtggaatatg
tacagatctt gttgctaact attcctgtga gtgcccaggc
gaatttatgg gaagaaattg tcaatacaaa tgctcaggcc
cactgggaat tgaaggtgga attatatcaa accagcaaat
cacagcttcc tctactcacc gagctctttt tggactccaa
aaatggtatc cctactatgc acgtcttaat aagaaggggc
ttataaatgc gtggacagct gcagaaaatg acagatggcc
gtggattcag ataaatttgc aaaggaaaat gagagttact
ggtgtgatta cccaaggagc caagaggatt ggaagcccag
agtatataaa atcctacaaa attgcctaca gtaatgatgg
aaagacttgg gcaatgtaca aagtgaaagg caccaatgaa
gacatggtgt ttcgtggaaa cattgataac aacactccat
atgctaactc tttcacaccc cccataaaag ctcagtatgt
aagactctat ccccaagttt gtcgaagaca ttgcactttg
cgaatggaac ttcttggctg tgaactgtcg ggttgttctg
agcctctggg tatgaaatca ggacatatac aagactatca
gatcactgcc tccagcatct tcagaacgct caacatggac
atgttcactt gggaaccaag gaaagctcgg ctggacaagc
aaggcaaagt gaatgcctgg acctctggcc acaatgacca
gtcacaatgg ttacaggtgg atcttcttgt tccaaccaaa
gtgactggca tcattacaca aggagctaaa gattttggtc
atgtacagtt tgttggctcc tacaaactgg cttacagcaa
tgatggagaa cactggactg tataccagga tgaaaagcaa
agaaaagata aggttttcca gggaaatttt gacaatgaca
ctcacagaaa aaatgtcatc gaccctccca tctatgcacg
acacataaga atccttcctt ggtcctggta cgggaggatc
acattgcggt cagagctgct gggctgcaca gaggaggaa
Mouse IgG1 Fc Amino Acid
SEQ ID NO: 3
DKIHTCPPCPAPELLGGPSVFLEPPKPKDILMISRIPEVICVVVDVS
HEDPEVKFNWYVDGVEVHNAKTKPREEQYNSTYRVVSVLIVLHQDWL
NGKEYKCKVSNKALPAPIEKTISKAKGQPREPQVYTLPPSRDELTKN
QVSLICLVKGFYPSDIAVEWESNGQPENNYKTIPPVLDSDGSFFLYS
KLIVDKSRWQQGNVFSCSVMHEALHNHYTQKSLSLSPGK
Mouse IgG1 Fc Amino Acid
SEQ ID NO: 4
Gataaaacccatacctgcccgccgtgcccggcgccggaactgctgggc
ggcccgagcgtgtttctgtttccgccgaaaccgaaagataccctgatg
attagccgcaccccggaagtgacctgcgtggtggtggatgtgagccat
gaagatccggaagtgaaatttaactggtatgtggatggcgtggaagtg
cataacgcgaaaaccaaaccgcgcgaagaacagtataacagcacctat
cgcgtggtgagcgtgctgaccgtgctgcatcaggattggctgaacggc
aaagaatataaatgcaaagtgagcaacaaagcgctgccggcgccgatt
gaaaaaaccattagcaaagcgaaaggccagccgcgcgaaccgcaggtg
tataccctgccgccgagccgcgatgaactgaccaaaaaccaggtgagc
ctgacctgcctggtgaaaggcttttatccgagcgatattgcggtggaa
tgggaaagcaacggccagccggaaaacaactataaaaccaccccgccg
gtgctggatagcgatggcagcttttttctgtatagcaaactgaccgtg
gataaaagccgctggcagcagggcaacgtgtttagctgcagcgtgatg
catgaagcgctgcataaccattatacccagaaaagcctgagcctgagc
ccgggcaaa
Del-1-Containing Amino Acid Sequence of
Expressed Product
SEQ ID NO: 5
MKRSVAVWLLVGLSLSVPQFGKGDICDPNPCENGGICLPGLADGSFSC
ECPDGFTDPNCSSVVEVASDEEEPTSAGPCIPNPCHNGGICEISEAYR
GDIFIGYVCKCPRGENGIHCQHNINECEVEPCKNGGICIDLVANYSCE
CPGEFMGRNCQYKCSGPLGIEGGIISNQQITASSTHRALFGLQKWYPY
YARLNKKGLINAWTAAENDRWPWIQINLQRKMRVTGVITQGAKRIGSP
EYIKSYKIAYSNDGKTWAMYKVKGINEDMVERGNIDNNTPYANSFTPP
IKAQYVRLYPQVCRRHCILRMELLGCELSGCSEPLGMKSGHIQDYQIT
ASSIFRILNMDMFTWEPRKARLDKQGKVNAWTSGHNDQSQWLQVDLLV
PIKVIGIITQGAKDFGHVQFVGSYKLAYSNDGEHWTVYQDEKQRKDKV
FQGNFDNDTHRKNVIDPPIYARHIRILPWSWYGRITLRSELLGCTEEE
AVPSSDPLQCGGIQDKTHTCPPCPAPELLGGPSVFLFPPKPKDTLMIS
RTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYT
LPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
K ARGGP EQKLISEEDLNSAVD HHHHHH
cDNA Sequence Encoding SEQ ID NO: 5
SEQ ID NO: 6
atgaagcgctc ggtagccgtc tggctcttgg tcgggctcag
cctcagtgtc ccccagttcg gcaaaggtga tatttgtgat
cccaatccat gtgaaaatgg aggtatctgt ttgccaggat
tggctgatgg ttccttttcc tgtgagtgtc cagatggctt
cacagacccc aactgttcta gtgttgtgga ggttgcatca
gatgaagaag aaccaacttc agcaggtccc tgcactccta
atccatgcca taatggagga acctgtgaaa taagtgaagc
ataccgaggg gatacattca taggctatgt ttgtaaatgt
ccccgaggat ttaatgggat tcactgtcag cacaacataa
atgaatgcga agttgagcct tgcaaaaatg gtggaatatg
tacagatctt gttgctaact attcctgtga gtgcccaggc
gaatttatgg gaagaaattg tcaatacaaa tgctcaggcc
cactgggaat tgaaggtgga attatatcaa accagcaaat
cacagcttcc tctactcacc gagctctttt tggactccaa
aaatggtatc cctactatgc acgtcttaat aagaaggggc
ttataaatgc gtggacagct gcagaaaatg acagatggcc
gtggattcag ataaatttgc aaaggaaaat gagagttact
ggtgtgatta cccaaggagc caagaggatt ggaagcccag
agtatataaa atcctacaaa attgcctaca gtaatgatgg
aaagacttgg gcaatgtaca aagtgaaagg caccaatgaa
gacatggtgt ttcgtggaaa cattgataac aacactccat
atgctaactc tttcacaccc cccataaaag ctcagtatgt
aagactctat ccccaagttt gtcgaagaca ttgcactttg
cgaatggaac ttcttggctg tgaactgtcg ggttgttctg
agcctctggg tatgaaatca ggacatatac aagactatca
gatcactgcc tccagcatct tcagaacgct caacatggac
atgttcactt gggaaccaag gaaagctcgg ctggacaagc
aaggcaaagt gaatgcctgg acctctggcc acaatgacca
gtcacaatgg ttacaggtgg atcttcttgt tccaaccaaa
gtgactggca tcattacaca aggagctaaa gattttggtc
atgtacagtt tgttggctcc tacaaactgg cttacagcaa
tgatggagaa cactggactg tataccagga tgaaaagcaa
agaaaagata aggttttcca gggaaatttt gacaatgaca
ctcacagaaa aaatgtcatc gaccctccca tctatgcacg
acacataaga atccttcctt ggtcctggta cgggaggatc
acattgcggt cagagctgct gggctgcaca gaggaggaa
gcggtaccgagctcggatccactccagtgtggtggaattcaag
ataaaacccatacctgcccgccgtgcccggcgccggaactgct
gggcggcccgagcgtgtttctgtttccgccgaaaccgaaagat
accctgatgattagccgcaccccggaagtgacctgcgtggtgg
tggatgtgagccatgaagatccggaagtgaaatttaactggta
tgtggatggcgtggaagtgcataacgcgaaaaccaaaccgcgc
gaagaacagtataacagcacctatcgcgtggtgagcgtgctga
ccgtgctgcatcaggattggctgaacggcaaagaatataaatg
caaagtgagcaacaaagcgctgccggcgccgattgaaaaaacc
attagcaaagcgaaaggccagccgcgcgaaccgcaggtgtata
ccctgccgccgagccgcgatgaactgaccaaaaaccaggtgag
cctgacctgcctggtgaaaggcttttatccgagcgatattgcg
gtggaatgggaaagcaacggccagccggaaaacaactataaaa
ccaccccgccggtgctggatagcgatggcagcttttttctgta
tagcaaactgaccgtggataaaagccgctggcagcagggcaac
gtgtttagctgcagcgtgatgcatgaagcgctgcataaccatt
atacccagaaaagcctgagcctgagcccgggcaaagctcgagg
agggcccgaacaaaaactcatctcagaagaggatctgaatagc
gccgtcgaccatcatcatcatcatcattga

Throughout this document, various references are mentioned. All such references are incorporated herein by reference, including the references set forth in the following list:

### REFERENCES

1. Ley, K., Laudanna, C., Cybulsky, M. I. & Nourshargh, S. Getting to the site of inflammation: the leukocyte adhesion cascade updated. Nat. Rev. Immunol. 7, 678-689 (2007).
2. Al-Rasheed, A., H. Scheerens, D. M. Rennick, H. M. Fletcher, and D. N. Tatakis. 2003. Accelerated alveolar bone loss in mice lacking interleukin-10. J Dent Res 82:632-635.
3. Armitage, G. C. (2002). Classifying periodontal diseases: a long-standing dilemma Periodontol. 2000 30, 9-23.
4. Armitage, G. C. Periodontal diagnoses and classification of periodontal diseases. Periodontol. 2000 34, 9-21 (2004).
5. Baker, P. J., Dixon, M., and Roopenian, D. C. (2000). Genetic control of susceptibility to Porphyromonas gingivalis-induced alveolar bone loss in mice. Infect. Immun 68, 5864-5868.
6. Brodlie, M. et al. Raised interleukin-17 is immunolocalised to neutrophils in cystic fibrosis lung disease. Eur. Respir. J. 37, 1378-1385 (2011).
7. Brown, L. J., B. A. Johns, and T. P. Wall. 2002. The economics of periodontal diseases. Periodontol 2000 29:223-234.
8. Cascao, R., Rosario, H. S., Souto-Carneiro, M. M., and Fonseca, J. E. (2010). Neutrophils in rheumatoid arthritis: More than simple final effectors. Autoimmun Rev. 9, 531-535.
9. Cevenini, E., Caruso, C., Candore, G., Capri, M., Nuzzo, D., Duro, G., Rizzo, C., Colonna-Romano, G., Lio, D., Di Carlo, D., et al. (2010). Age-related inflammation: the contribution of different organs, tissues and systems. How to face it for therapeutic approaches. Curr. Pharm. Des. 16, 609-618.
10. Chakravarti, A., Raquil, M. A., Tessier, P., and Poubelle, P. E. (2009). Surface RANKL of Toll-like receptor 4-stimulated human neutrophils activates osteoclastic bone resorption. Blood 114, 1633-1644.
11. Chavakis, E., Choi, E. Y., and Chavakis, T. (2009). Novel aspects in the regulation of the leukocyte adhesion cascade. Thromb Haemost 102, 191-197.
12. Choi, E. Y., E. Chavakis, M. A. Czabanka, H. F. Langer, L. Fraemohs, M. Economopoulou, R. K. Kundu, A. Orlandi, Y. Y. Zheng, D. A. Prieto, C. M. Ballantyne, S. L. Constant, W. C. Aird, T. Papayannopoulou, C. G. Gahmberg, M. C. Udey, P. Vajkoczy, T. Quertermous, S. Dimmeler, C. Weber, and T. Chavakis. 2008. Del-1, an endogenous leukocyte-endothelial adhesion inhibitor, limits inflammatory cell recruitment. Science 322:1101-1104.
13. Cua, D. J. & Tato, C. M. Innate IL-17-producing cells: the sentinels of the immune system. Nat. Rev. Immunol. 10, 479-489 (2010).
14. Darveau, R. P. Periodontitis: a polymicrobial disruption of host homeostasis. Nat. Rev. Microbiol. 8, 481-490 (2010).
15. Dababneh, R. et al. J Periodontol 79, 764-8 (2008).
16. Deas, D. E. et al. Periodontol 2000 32, 82-104 (2003).
17. Deban, L., Russo, R. C., Sironi, M., Moalli, F., Scanziani, M., Zambelli, V., Cuccovillo, I., Bastone, A., Gobbi, M., Valentino, S., et al. (2010). Regulation of leukocyte recruitment by the long pentraxin PTX3. Nat. Immunol. 11, 328-334.
18. Ding, Z. M., Babensee, J. E., Simon, S. I., Lu, H., Perrard, J. L., Bullard, D. C., Dai, X. Y., Bromley, S. K., Dustin, M. L., Entman, M. L., et al. (1999). Relative contribution of LFA-1 and Mac-1 to neutrophil adhesion and migration. J. Immunol. 163, 5029-5038.
19. Dorshkind, K., E. Montecino-Rodriguez, and R. A. Signer. 2009. The ageing immune system: is it ever too old to become young again? Nat Rev Immunol 9:57-62.
20. Dubin, P. J. & Kolls, J. K. Th17 cytokines and mucosal immunity. Immunol. Rev. 226, 160-171 (2008).
21. Eskan, M. A., et al. (2012) The leukocyte integrin antagonist Del-1 inhibits IL-17-mediated inflammatory bone loss. Nat Immunol 13, 465-473
22. Etzioni, A. & Tonetti, M. Immunol Rev 178, 138-47 (2000).
23. Eustace, A. et al. Identification of cells expressing IL-17A and IL-17F in the lungs of patients with COPD. Chest 139, 1089-1100 (2011).
24. Fan, et al. (2008) Brain Res.
25. Ferretti, S., Bonneau, O., Dubois, G. R., Jones, C. E. & Trifilieff, A. IL-17, produced by lymphocytes and neutrophils, is necessary for lipopolysaccharide-induced airway neutrophilia: IL-15 as a possible trigger. J. Immunol. 170, 2106-2112 (2003).
26. Franceschi, C., M. Bonafe, S. Valensin, F. Olivieri, M. De Luca, E. Ottaviani, and G. De Benedictis. 2000. Inflamm-aging. An evolutionary perspective on immunosenescence. Ann N Y Acad Sci 908:244-254.
27. Fransson, C., T. Berglundh, and J. Lindhe. 1996. The effect of age on the development of gingivitis. Clinical, microbiological and histological findings. J Clin Periodontol 23:379-385.
28. Gaffen, S. L. & Hajishengallis, G. A new inflammatory cytokine on the block: re-thinking periodontal disease and the Th1/Th2 paradigm in the context of Th17 cells and IL-17. J Dent Res 87, 817-828 (2008).
29. Gaffen, S. L. (2009a). The role of interleukin-17 in the pathogenesis of rheumatoid arthritis. Curr. Rheumatol. Rep. 11, 365-370.
30. Gaffen, S. L. (2009b). Structure and signalling in the IL-17 receptor family. Nat. Rev. Immunol. 9, 556-567.
31. Genco, R. J., and Van Dyke, T. E. (2010). Prevention: Reducing the risk of CVD in patients with periodontitis. Nat. Rev. Cardiol. 7, 479-480.
32. Gomez, C. R., Nomellini, V., Faunce, D. E. & Kovacs, E. J. Innate immunity and aging. Exp. Gerontol. 43, 718-728 (2008).
33. Graves, D. T., Fine, D., Teng, Y. T., Van Dyke, T. E. & Hajishengallis, G. The use of rodent models to investigate host-bacteria interactions related to periodontal diseases. J. Clin. Periodontol. 35, 89-105 (2008).
34. Grossman, et al., (2007) Am Heart J.
35. Haas, M. J., (2012) DELI: taking the bite out of periodontitis. SciBX: Science-Business eXchange. 5:15, 7-8.
36. Hajishengallis, G. 2009. Porphyromonas gingivalis-host interactions: open war or intelligent guerilla tactics? Microbes Infect 11:637-645.
37. Hajishengallis, G. 2009. Toll gates to periodontal host modulation and vaccine therapy. Periodontol 2000 51:in press.
38. Hajishengallis, G. 2010. Too old to fight? Aging and its toll on innate immunity. Mol. Oral Microbiol. 25:25-37.
39. Hajishengallis, G. et al. Low-abundance biofilm species orchestrates inflammatory periodontal disease through the commensal microbiota and complement. Cell Host Microbe 10, 497-506 (2011).
40. Hajishengallis, G., M.-A. K. Shakhatreh, M. Wang, and S. Liang. 2007. Complement receptor 3 blockade promotes IL-12-mediated clearance of Porphyromonas gingivalis and negates its virulence in vivo. J. Immunol. 179:2359-2367.
41. Happel, K. I., Zheng, M., Young, E., Quinton, L. J., Lockhart, E., Ramsay, A. J., Shellito, J. E., Schurr, J. R., Bagby, G. J., Nelson, S., et al. (2003). Cutting edge: roles of Toll-like receptor 4 and IL-23 in IL-17 expression in response to Klebsiella pneumoniae infection. J. Immunol. 170, 4432-4436.
42. Hartl, D., Krauss-Etschmann, S., Koller, B., Hordijk, P. L., Kuijpers, T. W., Hoffmann, F., Hector, A., Eber, E., Marcos, V., Bittmann, I., et al. (2008). Infiltrated neutrophils acquire novel chemokine receptor expression and chemokine responsiveness in chronic inflammatory lung diseases. J Immunol 181, 8053-8067.
43. Hasturk, H., Kantarci, A., Goguet-Surmenian, E., Blackwood, A., Andry, C., Serhan, C. N., and Van Dyke, T. E. (2007). Resolvin E1 regulates inflammation at the cellular and tissue level and restores tissue homeostasis in vivo. J. Immunol. 179, 7021-7029.
44. Hidai, C., Kawana, M., Kitano, H., and Kokubun, S. (2007). Discoidin domain of Dell protein contributes to its deposition in the extracellular matrix. Cell Tissue Res. 330, 83-95.
45. Hidai, et al. (1998) Genes Dev.
46. Ho, et al. (2004) Circulation.
47. Hoshino, A. et al. MPO-ANCA induces IL-17 production by activated neutrophils in vitro via classical complement pathway-dependent manner. J Autoimmun 31, 79-89 (2008).
48. Huttner, E. A., Machado, D. C., de Oliveira, R. B., Antunes, A. G. & Hebling, E. Effects of human aging on periodontal tissues. Spec. Care Dentist 29, 149-155 (2009).
49. Jeffcoat, M., et al. (2011) Periodontal infection and preterm birth: successful periodontal therapy reduces the risk of preterm birth. BJOG 118, 250-256.
50. Kanazawa, N., and F. Furukawa. 2007. Autoinflammatory syndromes with a dermatological perspective. J Dermatol 34:601-618.
51. Kasama, T., Y. Miwa, T. Isozaki, T. Odai, M. Adachi, and S. L. Kunkel. 2005. Neutrophil-derived cytokines: potential therapeutic targets in inflammation. Curr Drug Larbi, A., T. Fulop, and G. Pawelec. 2008 Immune receptor signaling, aging and autoimmunity. Adv Exp Med Biol 640:312-324.
52. Katsuragi, et al. (1994) J. Periodontol.
53. Kempf, T., Zarbock, A., Widera, C., Butz, S., Stadtmann, A., Rossaint, J., Bolomini-Vittori, M., Korf-Klingebiel, M., Napp, L. C., Hansen, B., et al. (2011). GDF-15 is an inhibitor of leukocyte integrin activation required for survival after myocardial infarction in mice. Nat. Med. 17, 581-588.
54. Kinane, D. F. & Hart, T. C. Crit Rev Oral Biol Med 14, 430-49 (2003).
55. Kolls, J. K., and Linden, A. (2004). Interleukin-17 family members and inflammation. Immunity 21, 467-476.
56. Lalla, E. & Papapanou, P. N. Diabetes mellitus and periodontitis: a tale of two common interrelated diseases. Nat. Rev. Endocrinol. 7, 738-748 (2011).
57. Lemos, H. P. et al. Prostaglandin mediates IL-23/IL-17-induced neutrophil migration in inflammation by inhibiting IL-12 and IFNg production. Proc. Natl. Acad. Sci. USA 106, 5954-5959 (2009).
58. Lester, S. R., Bain, J. L., Johnson, R. B., and Serio, F. G. (2007). Gingival concentrations of interleukin-23 and -17 at healthy sites and at sites of clinical attachment loss. J. Periodontol. 78, 1545-1550.
59. Ley, K., Laudanna, C., Cybulsky, M. I., and Nourshargh, S. (2007). Getting to the site of inflammation: the leukocyte adhesion cascade updated. Nat. Rev. Immunol. 7, 678-689.
60. Li, L. et al. IL-17 produced by neutrophils regulates IFN-γ-mediated neutrophil migration in mouse kidney ischemia-reperfusion injury. J. Clin. Invest. 120, 331-342 (2010).
61. Liang, S., Hosur, K. B., Domon, H. & Hajishengallis, G. Periodontal inflammation and bone loss in aged mice. J. Periodontal Res. 45, 574-578 (2010).
62. Lin, A. M. et al. Mast cells and neutrophils release IL-17 through extracellular trap formation in psoriasis. J. Immunol. 187, 490-500 (2011).
63. Lubberts, E. 2008. IL-17/Th17 targeting: on the road to prevent chronic destructive arthritis? Cytokine 41:84-91.
64. Lundberg, K., Wegner, N., Yucel-Lindberg, T., and Venables, P. J. (2010). Periodontitis in RA—the citrullinated enolase connection. Nat. Rev Rheumatol. 6, 727-730.
65. Lundqvist, C., Baranov, V., Teglund, S., Hammarstrom, S. & Hammarstrom, M. L. Cytokine profile and ultrastructure of intraepithelial gd T cells in chronically inflamed human gingiva suggest a cytotoxic effector function. J. Immunol. 153, 2302-2312 (1994).
66. Luster, A. D., Alon, R., and von Andrian, U. H. (2005) Immune cell migration in inflammation: present and future therapeutic targets. Nat Immunol 6, 1182-1190.
67. Martin, B., Hirota, K., Cua, D. J., Stockinger, B., and Veldhoen, M. (2009). Interleukin-17-producing gammadelta T cells selectively expand in response to pathogen products and environmental signals. Immunity 31, 321-330.
68. Meyle (1994) Periodontology.
69. Niederman, R., T. Westernoff, C. Lee, L. L. Mark, N. Kawashima, M. Ullman-Culler, F. E. Dewhirst, B. J. Paster, D. D. Wagner, T. Mayadas, R. O. Hynes, and P. Stashenko. 2001. Infection-mediated early-onset periodontal disease in P/E-selectin-deficient mice. J Clin Periodontol 28:569-575.
70. Nussbaum, G., and Shapira, L. (2011). How has neutrophil research improved our understanding of periodontal pathogenesis? J. Clin. Periodontol. 38, 49-59.
71. Ohyama, H., Kato-Kogoe, N., Kuhara, A., Nishimura, F., Nakasho, K., Yamanegi, K., Yamada, N., Hata, M., Yamane, J., and Terada, N. (2009). The involvement of IL-23 and the Th17 pathway in periodontitis. J. Dent. Res. 88, 633-638.
72. Orlova, V. V. et al. A novel pathway of HMGB1-mediated inflammatory cell recruitment that requires Mac-1-integrin. EMBO J. 26, 1129-1139 (2007).
73. Pawelec, G., and A. Larbi. 2008. Immunity and ageing in man: Annual Review 2006/2007. Exp Gerontol 43:34-38.
74. Pelletier, M., L. Maggi, A. Micheletti, E. Lazzeri, N. Tamassia, C. Costantini, L. Cosmi, C. Lunardi, F. Annunziato, S. Romagnani, and M. A. Cassatella. 2009. Evidence for a cross-talk between human neutrophils and Th17 cells. Blood.
75. Pihlstrom, B. L., Michalowicz, B. S. & Johnson, N. W. Periodontal diseases. Lancet 366, 1809-1820 (2005).
76. Penta, K., et al. (1999) Dell induces integrin signaling and angiogenesis by ligation of alphaVbeta3. J Biol Chem 274, 11101-11109.
77. Rajagopalan, et al, (2004) Hum Gene Ther.
78. Rittirsch, D., Flierl, M. A., and Ward, P. A. (2008). Harmful molecular mechanisms in sepsis. Nat. Rev. Immunol. 8, 776-787.
79. Serhan, C. N., Chiang, N., and Van Dyke, T. E. (2008). Resolving inflammation: dual anti-inflammatory and pro-resolution lipid mediators. Nat. Rev. Immunol. 8, 349-361.
80. Seymour, G. J., P. J. Ford, M. P. Cullinan, S. Leishman, and K. Yamazaki. 2007. Relationship between periodontal infections and systemic disease. Clin Microbiol Infect 13 Suppl 4:3-10.
81. Simpson, J. L., S. Phipps, and P. G. Gibson. 2009. Inflammatory mechanisms and treatment of obstructive airway diseases with neutrophilic bronchitis. Pharmacol Ther 124:86-95.
82. Smith, E. et al. IL-17A inhibits the expansion of IL-17A-producing T cells in mice through “short-loop” inhibition via IL-17 receptor. J. Immunol. 181, 1357-1364 (2008).
83. Sollecito, T. P. et al. Med Oral Patol Oral Cir Bucal 10, 142-50 (2005).
84. Stark, M. A., Huo, Y., Burcin, T. L., Morris, M. A., Olson, T. S., and Ley, K. (2005). Phagocytosis of apoptotic neutrophils regulates granulopoiesis via IL-23 and IL-17. Immunity 22, 285-294.
85. Tonetti, M. S., F. D'Aiuto, L. Nibali, A. Donald, C. Storry, M. Parkar, J. Suvan, A. D. Hingorani, P. Vallance, and J. Deanfield. 2007. Treatment of periodontitis and endothelial function. N Engl J Med 356:911-920.
86. U.S. Patent Application Publication No. 2003/0100889.
87. U.S. Patent Application Publication No. 2009/0209630
88. U.S. Patent Application Publication No. 2011/0136894.
89. U.S. Patent Publication No. 2007/0248641.
90. van der Velden, U. 1991. The onset age of periodontal destruction. J Clin Periodontol 18:380-383.
91. Van Dyke, T. E., and C. N. Serhan. 2003. Resolution of inflammation: a new paradigm for the pathogenesis of periodontal diseases. J. Dent. Res. 82:82-90.
92. Waldrop, T. C. et al. J Periodontol 58, 400-16 (1987).
93. Wang, M., M.-A. K. Shakhatreh, D. James, S. Liang, S.-i. Nishiyama, F. Yoshimura, D. R. Demuth, and G. Hajishengallis. 2007. Fimbrial proteins of Porphyromonas gingivalis mediate in vivo virulence and exploit TLR2 and complement receptor 3 to persist in macrophages. J. Immunol. 179:2349-2358.
94. Weitz-Schmidt, G., Welzenbach, K., Dawson, J. & Kallen, J. Improved lymphocyte function-associated antigen-1 (LFA-1) inhibition by statin derivatives: molecular basis determined by x-ray analysis and monitoring of LFA-1 conformational changes in vitro and ex vivo. J. Biol. Chem. 279, 46764-46771 (2004).
95. Witowski, J. et al. IL-17 stimulates intraperitoneal neutrophil infiltration through the release of GROa chemokine from mesothelial cells. J. Immunol. 165, 5814-5821 (2000).
96. Wolf, R. et al. Gene from a psoriasis susceptibility locus primes the skin for inflammation. Sci. Transl. Med. 2, 61ra90 (2010).
97. Ye, P., Rodriguez, F. H., Kanaly, S., Stocking, K. L., Schurr, J., Schwarzenberger, P., Oliver, P., Huang, W., Zhang, P., Zhang, J., et al. (2001). Requirement of interleukin 17 receptor signaling for lung CXC chemokine and granulocyte colony-stimulating factor expression, neutrophil recruitment, and host defense. J. Exp. Med. 194, 519-527.
98. Yu, J. J., Ruddy, M. J., Wong, G. C., Sfintescu, C., Baker, P. J., Smith, J. B., Evans, R. T., and Gaffen, S. L. (2007). An essential role for IL-17 in preventing pathogen-initiated bone destruction: recruitment of neutrophils to inflamed bone requires IL-17 receptor-dependent signals. Blood 109, 3794-3802.
99. Zhong, et al. (2003) J Clin Invest.

1. A method for treating periodontitis in a subject, comprising: administering to the subject a Del-1 polypeptide.

2. The method of claim 1, and further comprising monitoring the subject for periodontal inflammation and/or bone loss.

3. The method of claim 1, and further comprising monitoring one or more markers associated with periodontal inflammation and/or bone loss.

4. The method of claim 3, wherein the one or more markers comprises at least one marker selected from: IL-1β and MMP-8.

5. The method of claim 1, and further comprising identifying a subject having periodontitis or a risk thereof.

6. The method of claim 1, wherein the subject is identified as having periodontal inflammation and/or bone loss, a condition associated with neutrophil dysfunction, or a condition selected from: a leukocyte adhesion deficiency (LAD), Wiskott-Aldrich syndrome, Chediak-Higashi syndrome, lazy leukocyte syndrome, Papillon-LeFevre syndrome, Down's syndrome, congenital agranulocytosis, cyclic netropenia (idiopathic or drug-induced), autoimmune neutropenia, chronic idiopathic neutropenia, HIV-associated neutropenia, and neutropenia in cancer patients under chemotherapy and/or radiation therapy.

7-8. (canceled)

9. The method of claim 1, wherein the subject is identified as being at least 30 years old.

10. (canceled)

11. The method of claim 1, wherein the Del-1 polypeptide is administered to a gingiva or to a periodontal pocket of the subject.

12. (canceled)

13. The method of claim 1, wherein the Del-1 polypeptide is administered periodically.

14-15. (canceled)

16. The method of claim 1, wherein the Del-1 polypeptide is administered periodically during a treatment period lasting at least 1 month.

17. A composition, comprising a Del-1 polypeptide, wherein the Del-1 polypeptide is formulated for delivery to an oral cavity of a subject.

18. (canceled)

19. The composition of claim 17, wherein the Del-1 polypeptide is provided as a fusion protein.

20. The composition of claim 17, wherein the Del-1 polypeptide is provided as a fusion protein that includes an IgG Fc fragment.

21. The composition of claim 17, wherein the Del-1 polypeptide is provided in a toothpaste, a mouthwash, a chewing gum, a dental floss, a beverage, a food product, a gel, a slow-release gel, a tablet, a granule, a film, or a thin film of biodegradable matrix.

22. (canceled)

23. The composition of claim 17, wherein the Del-1 polypeptide is expressed by a cell containing a nucleic acid encoding the Del-1 polypeptide.

24. The method of claim 1, wherein the Del-1 polypeptide is expressed by a cell containing a nucleic acid encoding the Del-1 polypeptide and the cell is administered to the subject.

25. (canceled)

26. A kit, comprising a Del-1 polypeptide and/or the composition of claim 17; and a device for administering the Del-1 polypeptide or composition.

27-28. (canceled)

29. The kit of claim 26, comprising multiple doses of the Del-1 polypeptide and/or the composition of claim 17.

30-33. (canceled)

34. The method of claim 1, wherein the Del-1 polypeptide is provided as a fusion protein.

35. The method of claim 34, wherein the Del-1 polypeptide is provided as a fusion protein that includes an IgG Fc fragment.


SEQUENCE LISTING (6 sequences)

SEQ ID NO: 1 - protein, 480 aa, Homo sapiens
MKRSVAVWLLVGLSLSVPQFGKGDICDPNPCENGGICLPGLADGSFSCECPDGFTDPNCSSVVEVASDEEEPTSAGPCTPNPCHNGGTCEISEAYRGDTFIGYVCKCPRGFNGIHCQHNINECEVEPCKNGGICTDLVANYSCECPGEFMGRNCQYKCSGPLGIEGGIISNQQITASSTHRALFGLQKWYPYYARLNKKGLINAWTAAENDRWPWIQINLQRKMRVTGVITQGAKRIGSPEYIKSYKIAYSNDGKTWAMYKVKGTNEDMVFRGNIDNNTPYANSFTPPIKAQYVRLYPQVCRRHCTLRMELLGCELSGCSEPLGMKSGHIQDYQITASSIFRTLNMDMFTWEPRKARLDKQGKVNAWTSGHNDQSQWLQVDLLVPTKVTGIITQGAKDFGHVQFVGSYKLAYSNDGEHWTVYQDEKQRKDKVFQGNFDNDTHRKNVIDPPIYARHIRILPWSWYGRITLRSELLGCTEEE

SEQ ID NO: 2 - DNA, 1440 bp, Homo sapiens
atgaagcgctcggtagccgtctggctcttggtcgggctcagcctcagtgtcccccagttcggcaaaggtgatatttgtgatcccaatccatgtgaaaatggaggtatctgtttgccaggattggctgatggttccttttcctgtgagtgtccagatggcttcacagaccccaactgttctagtgttgtggaggttgcatcagatgaagaagaaccaacttcagcaggtccctgcactcctaatccatgccataatggaggaacctgtgaaataagtgaagcataccgaggggatacattcataggctatgtttgtaaatgtccccgaggatttaatgggattcactgtcagcacaacataaatgaatgcgaagttgagccttgcaaaaatggtggaatatgtacagatcttgttgctaactattcctgtgagtgcccaggcgaatttatgggaagaaattgtcaatacaaatgctcaggcccactgggaattgaaggtggaattatatcaaaccagcaaatcacagcttcctctactcaccgagctctttttggactccaaaaatggtatccctactatgcacgtcttaataagaaggggcttataaatgcgtggacagctgcagaaaatgacagatggccgtggattcagataaatttgcaaaggaaaatgagagttactggtgtgattacccaaggagccaagaggattggaagcccagagtatataaaatcctacaaaattgcctacagtaatgatggaaagacttgggcaatgtacaaagtgaaaggcaccaatgaagacatggtgtttcgtggaaacattgataacaacactccatatgctaactctttcacaccccccataaaagctcagtatgtaagactctatccccaagtttgtcgaagacattgcactttgcgaatggaacttcttggctgtgaactgtcgggttgttctgagcctctgggtatgaaatcaggacatatacaagactatcagatcactgcctccagcatcttcagaacgctcaacatggacatgttcacttgggaaccaaggaaagctcggctggacaagcaaggcaaagtgaatgcctggacctctggccacaatgaccagtcacaatggttacaggtggatcttcttgttccaaccaaagtgactggcatcattacacaaggagctaaagattttggtcatgtacagtttgttggctcctacaaactggcttacagcaatgatggagaacactggactgtataccaggatgaaaagcaaagaaaagataaggttttccagggaaattttgacaatgacactcacagaaaaaatgtcatcgaccctcccatctatgcacgacacataagaatccttccttggtcctggtacgggaggatcacattgcggtcagagctgctgggctgcacagaggaggaa

SEQ ID NO: 3 - protein, 227 aa, Mus musculus
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 681 bp, Mus musculus
gataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 5 - protein, 747 aa, Artificial Sequence
  Synthetic
MKRSVAVWLLVGLSLSVPQFGKGDICDPNPCENGGICLPGLADGSFSCECPDGFTDPNCSSVVEVASDEEEPTSAGPCTPNPCHNGGTCEISEAYRGDTFIGYVCKCPRGFNGIHCQHNINECEVEPCKNGGICTDLVANYSCECPGEFMGRNCQYKCSGPLGIEGGIISNQQITASSTHRALFGLQKWYPYYARLNKKGLINAWTAAENDRWPWIQINLQRKMRVTGVITQGAKRIGSPEYIKSYKIAYSNDGKTWAMYKVKGTNEDMVFRGNIDNNTPYANSFTPPIKAQYVRLYPQVCRRHCTLRMELLGCELSGCSEPLGMKSGHIQDYQITASSIFRTLNMDMFTWEPRKARLDKQGKVNAWTSGHNDQSQWLQVDLLVPTKVTGIITQGAKDFGHVQFVGSYKLAYSNDGEHWTVYQDEKQRKDKVFQGNFDNDTHRKNVIDPPIYARHIRILPWSWYGRITLRSELLGCTEEEAVPSSDPLQCGGIQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKARGGPEQKLISEEDLNSAVDHHHHHH

SEQ ID NO: 6 - DNA, 2244 bp, Artificial
  Synthetic
atgaagcgctcggtagccgtctggctcttggtcgggctcagcctcagtgtcccccagttcggcaaaggtgatatttgtgatcccaatccatgtgaaaatggaggtatctgtttgccaggattggctgatggttccttttcctgtgagtgtccagatggcttcacagaccccaactgttctagtgttgtggaggttgcatcagatgaagaagaaccaacttcagcaggtccctgcactcctaatccatgccataatggaggaacctgtgaaataagtgaagcataccgaggggatacattcataggctatgtttgtaaatgtccccgaggatttaatgggattcactgtcagcacaacataaatgaatgcgaagttgagccttgcaaaaatggtggaatatgtacagatcttgttgctaactattcctgtgagtgcccaggcgaatttatgggaagaaattgtcaatacaaatgctcaggcccactgggaattgaaggtggaattatatcaaaccagcaaatcacagcttcctctactcaccgagctctttttggactccaaaaatggtatccctactatgcacgtcttaataagaaggggcttataaatgcgtggacagctgcagaaaatgacagatggccgtggattcagataaatttgcaaaggaaaatgagagttactggtgtgattacccaaggagccaagaggattggaagcccagagtatataaaatcctacaaaattgcctacagtaatgatggaaagacttgggcaatgtacaaagtgaaaggcaccaatgaagacatggtgtttcgtggaaacattgataacaacactccatatgctaactctttcacaccccccataaaagctcagtatgtaagactctatccccaagtttgtcgaagacattgcactttgcgaatggaacttcttggctgtgaactgtcgggttgttctgagcctctgggtatgaaatcaggacatatacaagactatcagatcactgcctccagcatcttcagaacgctcaacatggacatgttcacttgggaaccaaggaaagctcggctggacaagcaaggcaaagtgaatgcctggacctctggccacaatgaccagtcacaatggttacaggtggatcttcttgttccaaccaaagtgactggcatcattacacaaggagctaaagattttggtcatgtacagtttgttggctcctacaaactggcttacagcaatgatggagaacactggactgtataccaggatgaaaagcaaagaaaagataaggttttccagggaaattttgacaatgacactcacagaaaaaatgtcatcgaccctcccatctatgcacgacacataagaatccttccttggtcctggtacgggaggatcacattgcggtcagagctgctgggctgcacagaggaggaagcggtaccgagctcggatccactccagtgtggtggaattcaagataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaagctcgaggagggcccgaacaaaaactcatctcagaagaggatctgaatagcgccgtcgaccatcatcatcatcatcattga